(19) United States
(12) Patent Application Publication
(10) Pub. No.: US 2010/0069304 A1
(43) Pub. Date: Mar. 18, 2010

(54) PROTECTIVE EFFECTS OF INHIBITING THE INTERACTION OF CALMODULIN AND MUTANT HUNTINGTIN PROTEIN

(76) Inventor: Nancy A. Muma, Lawrence, KS (US)

Correspondence Address: Workman Nydegger;1000 Eagle Gate Tower, 60 East South Temple, Salt Lake City, UT, 84111, US

(21) Appl. No.: 12/561,167

(22) Filed: Sep. 16, 2009

Related U.S. Application Data
(60) Provisional application No. 61/097,442, filed on Sep. 16, 2008.

(51) Int. Cl. A61K 38/16; C07K 14/435; C12N 15/11; C12N 7/00; C12N 5/06; G01N 33/68; C12Q 1/02; C12Q 1/48

(52) U.S. Cl. 514/12; 530/324; 536/23.1; 435/235.1; 435/375; 436/86; 435/29; 435/15

(57) ABSTRACT

An artificial polypeptide can be used in a treatment for Huntington's disease. The inventive polypeptide sequence is capable of interacting with mutant huntingtin so as to inhibit interactions between mutant huntingtin or a fragment of mutant huntingtin and calmodulin. The inventive polypeptide sequence can be a portion of calmodulin described herein or an analog or derivative thereof that binds with the polyglutamate portion of a mutant huntingtin protein. For example, polypeptide sequence can include a sequence of KDTDSEEEIREAFRVFDKDGNGYISAAELRHVMTNLGEKLTDEEV (SEQ ID NO: 1) or a portion thereof analog thereof or derivative thereof.

### CROSS-REFERENCE TO RELATED APPLICATIONS

This U.S. patent application claims benefit of U.S. provisional patent application having Ser. No. 61/097,442, filed on Sep. 16, 2008, which provisional application is incorporated herein by specific reference in its entirety.

### BACKGROUND OF THE INVENTION

Huntington disease (HD) is an autosomal dominant neurodegenerative disease caused by an unstable CAG trinucleotide-repeat in the gene encoding the huntingtin protein. Amino-terminal fragments of huntingtin with an expanded glutamine repeat form insoluble deposits in neurons in these brain regions. Huntingtin with an expanded glutamine repeat is a substrate of transglutaminase (TG) and as the length of the glutamine repeat is increased, the enzyme activity increases. It has been hypothesized that TG modifies huntingtin thereby aiding in the formation/stabilization of huntingtin containing aggregates and stabilization of monomeric huntingtin.

Transglutaminases are a family of calcium-dependent enzymes. TG can catalyze the formation of a ε-(γ-glutamyl)lysine bond between the γ-carboxy group of a peptide-bound glutamine and the &amino group of a peptide-bound lysine or a poly- or monoamine. Numerous lines of evidence implicate the involvement of TG in HD. TG mRNA, protein levels, and activity have all been shown to be elevated in HD brain. Our laboratory has shown that ε-(γ-glutamyl)lysine bonds and TG colocalize with huntingtin in intranuclear inclusions in HD. Furthermore, treating HD transgenic mice with cystamine, a TG inhibitor, and knock-out of TG both increase survival. In cells expressing mutant huntingtin and TG, treatment with cystamine increased cell survival and decreased the cross-linking of huntingtin.

Calmodulin (CaM), a calcium (Ca²⁺) binding protein that activates many enzymes, has been shown to interact with TG and increase TG activity as well as interact with mutant huntingtin. We previously demonstrated that CaM colocalizes with TG2 and with huntingtin in inclusions in HD cortex. Inhibition of CaM results in decreased TG-catalyzed modifications of huntingtin in cells expressing mutant huntingtin and TG2. It has also been shown that mutant huntingtin with an expanded glutamine repeat interacts with CaM with a higher affinity than wild-type huntingtin. In HD, mutant huntingtin may interact with both CaM and TG resulting in an increase in the interaction between CaM and TG and a subsequent increase in TG activity. CaM and TG interacting with mutant huntingtin may result in the proteins being sequestered into aggregates, thereby hindering normal functions of both CaM and TG.

Therefore, it would be advantageous to inhibit interactions between mutant huntingtin and other proteins. Additionally, it would be beneficial to inhibit interactions between mutant huntingtin and other proteins such as CaM and TG without adversely affecting the functionality of CaM and TG to perform normal physiological functions.

### BRIEF SUMMARY OF THE INVENTION

In one embodiment, the present invention includes an artificial polypeptide that has a polypeptide sequence having an amino acid sequence of a portion of calmodulin or analog or derivative thereof. The inventive polypeptide sequence is capable of interacting with mutant huntingtin so as to inhibit interactions between mutant huntingtin and calmodulin. The inventive polypeptide sequence can be a portion of calmodulin described herein or an analog or derivative thereof that binds with the polyglutamate portion of a mutant huntingtin protein.

In one embodiment, the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof. Also, the polypeptide having SEQ ID NO: 1 can be truncated by up to 15 amino acids from the N-terminus or the C-terminus.

In one embodiment, the polypeptide that binds with mutant huntingtin consists of SEQ ID NO: 1.

In one embodiment, the present invention includes a pharmaceutical composition that includes the artificial polypeptide sequence that binds with mutant huntingtin and a pharmaceutically acceptable carrier.

In one embodiment, the present invention includes a nucleic acid that encodes for the artificial polypeptide that binds with mutant huntingtin.

In one embodiment, the present invention includes a cell having a mutant huntingtin protein bound to the artificial polypeptide described herein. Such binding inhibiting calmodulin in the cell from interacting with the mutant huntingtin protein.

In one embodiment, the present invention can include a method of treating, inhibiting, and/or preventing Huntington's disease or symptoms thereof. Such a method can include providing a polypeptide sequence being capable of interacting with mutant huntingtin so as to inhibit interactions between mutant huntingtin and calmodulin; and administering the polypeptide sequence to a subject having or susceptible to Huntington's Disease. The polypeptide sequence can be any sequence described herein that is capable of interacting with mutant huntingtin so as to inhibit mutant huntingtin from interacting with calmodulin.

In one embodiment, the present invention can include a method of inhibiting calmodulin from interacting with a mutant huntingtin protein. Such a method can include: providing a mutant huntingtin protein having a polyglutamine sequence in the presence of a calmodulin protein; and contacting the mutant huntingtin protein with a polypeptide, said polypeptide sequence being capable of interacting with the mutant huntingtin so as to inhibit interactions between the mutant huntingtin and calmodulin proteins. In one aspect, the polypeptide is a portion of the calmodulin protein or analog or derivative thereof. For example, the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof.

In one embodiment, a substance, such as a small molecule, peptide mimetic, or other substance capable of being used as a drug can be employed to disrupt biding between CaM and mutant huntingtin. The experimental protocols described herein can be employed with substances to determine whether or not a substance can inhibit the binding. Accordingly, a drug screen can be performed that tests compounds from a compound library to determine whether or not any compound or substance can function to inhibit CaM from binding with mutant huntingtin. As such, the present invention covers screening methods to find any compound capable of disrupting the interaction between mutant huntingtin and CaM, and any of the compounds that are found.

In one embodiment, the polypeptide binds to the mutant huntingtin protein and inhibits the mutant huntingtin protein from interacting with a transglutaminase.

In one embodiment, the mutant huntingtin protein is located within a cell, such as a neuronal cell.

In one embodiment, the mutant huntingtin protein is located within a subject having Huntington's disease.

In one embodiment, the present invention includes a method for screening for a substance that inhibits mutant huntingtin from interacting with calmodulin. Such a method can include: providing a mutant huntingtin protein or a mutated portion thereof having a polyglutamine; contacting the mutant huntingtin protein or mutated portion thereof with a substance; and determining whether or not the substance inhibits the mutant huntingtin protein or mutated portion thereof from interacting with the calmodulin protein. In one aspect, the contacting can be conducted within a cell. In one aspect, the determining can include contacting the mutant huntingtin protein or mutated portion thereof with calmodulin-agarose.

In one embodiment, the screening method can further include providing a cell expressing the mutant huntingtin or mutated portion thereof. The cell can also express a transglutaminase.

In one embodiment, the screening method can further include: lysing the cell; and mixing the cell lysate with calmodulin-agarose.

In one embodiment, the present invention provides a method for inhibiting transglutaminase from modifying a mutant huntingtin protein. Such a method can include: providing a mutant huntingtin protein having a polyglutamine sequence; and contacting the mutant huntingtin protein with a substance capable of interacting with the mutant huntingtin so as to inhibit a transglutaminase from modifying the mutant huntingtin. The substance can be a small molecule, drug, or an artificial polypeptide sequence, such as a sequence having an amino acid sequence of a portion of calmodulin or analog or derivative thereof or an entirely different sequence.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is a schematic representation of calmodulin constructs depicting the protein region of wild-type calmodulin included in each construct (calcium binding sites are underlined). FIG. 1 discloses the full length calmodulin sequence as SEQ ID NO: 29 and the construct regions (disclosed in black font) as SEQ ID NOS 30-32 and 1, respectively, in order of appearance.

FIG. 2A is a western blot of protein immunoprecipitated using the 81D4 antibody, (for the TG-catalyzed bond) conjugated to sepharose and then examined on a western blot to detect the levels of transglutaminase-modified huntingtin protein.

FIG. 2B is a graph showing the levels of TG-modified huntingtin in the cells expressing N-terminal huntingtin containing an expanded glutamine domain (H), TG 2 (T), and one of the CaM constructs depicted in FIG. 1. CaM-center and CaM-C-term significantly reduced the ability of transglutaminase to modify mutant huntingtin protein compared to cells not treated with one of the CaM constructs.

FIG. 2C is a western blot showing equal expression of total mutant huntingtin and total transglutaminase 2 (TG2) in all groups.

FIG. 2D is a western blot of protein immunoprecipitated using the 81D4 antibody, (for the TG-catalyzed bond) conjugated to sepharose and then examined on a western blot to detect the levels of transglutaminase-modified huntingtin protein. The lanes shown in FIG. 2D are the same as shown in FIG. 2F.

FIG. 2E is a graph showing the levels of TG-modified huntingtin in the cells expressing H, T, and CaM polypeptide constructs. The CaM-overlap, CaM-C-term, and CaM-center polypeptides significantly reduced the ability of TG to modify mutant huntingtin protein compared to cells expressing H, T, and V (control).

FIG. 2F is a western blot showing equal expression of total huntingtin and total transglutaminase 2 (TG2) in all groups.

FIG. 3A is a graph showing the levels of cytotoxicity in the cells expressing N-terminal huntingtin containing an expanded glutamine domain (H), TG 2 (T), and a CaM-fragment polypeptide. CaM-center and CaM-C-term significantly reduced cytoxicity compared to cells expressing H, T and vector (V) (control).

FIG. 3B is a graph showing that the level of cytotoxicity in the cells expressing H, T, and CaM-overlap were significantly lower than in cells expressing H, T, and V (control) but not significantly different than cells not expressing mutant huntingtin (TV).

FIGS. 4A-4B are graphs showing the increased release of intracellular Ca²⁺-associated with mutant huntingtin expression was attenuated in the cells expressing H, T, and CaM-center compared to cells expressing H, T, and V (control). Furthermore, cells expressing H, T, and CaM-center had a significantly smaller increase in Ca²⁺ similar to the increase in TV transfected cells.

FIG. 5A is an immunoblot analysis indicating that there was variation in the level of expression of the CaM constructs in the experiment depicted in 4A, however the expression levels of the CaM-constructs do not appear to play a significant role in their effects, since CaM-C-term and CaM-center had similar beneficial effects but immunoblot analysis indicated disparate expression levels.

FIG. 5B are images that show a similar percentage and staining intensity for all 4 CaM constructs which were also all expressed in the cytoplasm as well as the nucleus of cells. HEK-293T cells were either untransfected (control) or transfected with either CaM-N-term, CaM-C-term, CaM-center or CaM-overlap. Cells were stained for the CaM constructs which appear red (gray in right and left columns in grayscale) and nuclei were visualized using DAPI which appear blue (gray in center column in grayscale).

FIG. 6A is a representative western blot demonstrating the ability of the CaM-overlap polypeptide to disrupt the binding of mutant huntingtin protein to CaM. The amount of mutant huntingtin bound to CaM in the absence (HTV) and presence (HTCaM-overlap) the CaM-overlap peptide are shown in the second and third lane respectively.

FIG. 6B is a graph showing the quantification of repeated western blots for which 6A is an example.

FIG. 6C is a representative immunoblot of total huntingtin levels in cell lysates from the same western blot depicted in 6A.

FIG. 7A shows that SHSY5Y cells were infected with varying MOIs (0, 0.01, 0.1, 1, 5, 10, 50, 75, 100) of AAV-GFP. Forty-eight hours post-infection cells were assessed for percent AAV-mediated GFP expression by flow cytometry.

FIG. 7B are images that show differentiated SHSY5Y were infected with either AAV-CaM-peptide, AAV-scram-CaM-peptide, or AAV-GFP (MOI=50). Forty-eight hours post-infection cells were examined with fluorescence microscopy to examine AAV-mediated GFP expression.

FIG. 8A is a western blot analysis of human neuroblastoma SHSY5Y cells stably expressing N-terminal mutant huntingtin. SHSY5Y cells were transfected with N-terminal mutant huntingtin with an expanded polyglutamine repeat (SHSY5Y-htt-N63-148Q cells) and were selected based on their resistance to blasticidin. Blot was probed for N-terminal mutant huntingtin (upper) and actin (lower).

FIG. 8B shows non-htt-SHSY5Y cells and SHSY5Y-htt-N63-148Q cells that were treated with 10 μM retinoic acid for 4 days and then infected with either AAV-CaM-peptide, AAV-scram-CaM-peptide, or AAV-GFP (MOI=50). Forty-eight hours post-infection cells were assessed for percent AAV-mediated GFP expression by flow cytometry.

FIGS. 9A-9B show the level of TG-modified N-terminal mutant huntingtin in the SHSY5Y-htt-N63-148Q cells expressing CaM-peptide was significantly lower than in SHSY5Y-htt-N63-148Q cells expressing GFP (control) or scram-CaM-peptide. FIG. 9A is a graph that shows quantification of several repetitions of the experiment depicted in the immunoblot in 9B. FIG. 9B is a representative immunoblot of protein immunoprecipitated using the 81D4 antibody (for TG-catalyzed bonds) conjugated to sepharose and then probed to detect transglutaminase-modified N-terminal mutant huntingtin.

FIG. 10 is a graph that shows the level of cytotoxicity in SHSY5Y-htt-N63-148Q cells expressing CaM-peptide was significantly lower than in SHSY5Y-htt-N63-148Q cells expressing GFP (control) or scram-CaM-peptide. LDH levels were measured and used as an index of cytotoxicity.

FIGS. 11A-11B show there were no significant differences in total ex vivo or in situ TG activity in non-htt-SHSY5Y cells or SHSY5Y-htt-N63-148Q cells expressing CaM-peptide, scram-CaM-peptide or GFP. FIG. 11A is a graph that shows there were no significant differences in total ex vivo TG activity in non-htt-SHSY5Y cells expressing either CaM-peptide, scram-CaM-peptide or GFP (control). There was a small but insignificant increase in total ex vivo TG activity in SHSY5Y-htt-N63-148Q cells expressing scram-CaM-peptide or GFP compared to non-htt-SHSY5Y cells and SHSY5Y-htt-N63-148Q cells expressing CaM-peptide. FIG. 11B is a graph that shows there were no significant differences in total in situ TG activity in non-htt-SHSY5Y cells expressing CaM-peptide, scram-CaM-peptide or GFP. However, total in situ TG activity was significantly less in non-htt-SHSY5Y cells compared to SHSY5Y-N63-htt-148Q cells expressing scram-CaM-peptide or GFP. But, total in situ TG activity in non-htt SH-SY5Y cells was not significantly different from total TG activity in SH-SY5Y-htt-N63-148Q cells expressing the CaM-peptide.

FIG. 12A shows the expression of CaM-peptide in N-terminal mutant huntingtin expressing cells does not significantly affect CaM kinase II activity. SH-SY5Y cells were transfected with vector (V), htt-N63-148Q (H)+V, CaM-peptide+V, or H+CaM-peptide. Cells were harvested 48 hours post transfection and CaM kinase II activity was evaluated by the SigmaTECT CaM kinase II Assay System as per the manufacturer's directions.

FIG. 12B shows the CaM kinase II assay was also performed in the absence of exogenous calmodulin. Data shown are the mean IOD±SEM. Two-way ANOVA indicates a significant main effect of EGTA (F_{(1,16)}=143.37, p<0.0001). However, there was no significant main effect of transfection on CaM kinase II activity (F_{(3,16)}=0.86, p=0.48). The interaction between EGTA and transfection was also not significant (F_{(3,16)}=1.89, p=0.17). Newman-Keuls multiple comparison test indicates no significant differences in CaM kinase II activity among the various transfections in either the presence or absence of EGTA.

FIG. 12C: the top gel shows protein expression of CaM kinase II remained at the same level after transfections of either htt-N-148Q (H), CaM-peptide or the combination of those two constructs; the middle gel shows myc antibody was used to confirm the expression of htt-N-148Q; and the bottom gel shows equal loading was verified by reprobing the same membrane with an actin antibody.

FIGS. 13A-13D show interaction of CaM and mutant huntingtin in vitro. The western blots show the level of CaM-bound to huntingtin-exon 1 with an expanded polyglutamine repeat (htt-exon1-44Q) in the absence and presence of varying concentrations of CaM-peptide. The amount of CaM-bound htt-exon1-44Q was significantly lower when 10 μM of CaM-peptide was present than when no CaM peptide was present (control). Furthermore, lower concentrations of CaM peptide are required to inhibit self-aggregated htt-exon1-44Q as shown at the top of the blot marked with as the stacking gel. However, 10 μM of CaM-peptide did not affect the binding of CaM with calcineurin, a known CaM-binding protein. FIG. 13A is a representative western blot of protein immunoprecipitated using CaM-agarose. Blot was probed for N-terminal mutant huntingtin. FIG. 13B shows quantification of repeated immunoblots of which the blots in FIG. 13A is a representative example. FIG. 13C is a representative western blot of protein immunoprecipitated using CaM-agarose. Blot was probed for calcineurin. FIG. 13D shows quantification of repeated immunoblots of which the blot in FIG. 13C is a representative example.

FIGS. 14A-14B show the level of CaM-bound huntingtin-exon 1 with an expanded polyglutamine repeat (htt-exon1-44Q) in the absence and presence of varying concentrations of CaM-peptide and/or W-5. The presence of W-5 did not alter the amount of CaM-bound htt-exon1-44Q. The amount of CaM-bound htt-exon1-44Q was significantly lower when 10 μM of CaM-peptide was present than when W-5 or no CaM peptide was present (control). However, the amount of CaM-bound htt-exon1-44Q was significantly increased when 664 μM W-5 is present along with 10 μM of CaM-peptide. FIG. 14A is a representative western blot of protein immunoprecipitated using CaM-agarose. Blot was probed for N-terminal mutant huntingtin. FIG. 14B shows quantification of immunoblot of FIG. 14A depicted graphically.

FIGS. 15A-15B show the effect of CaM-fragment on body weight and survival. FIG. 15A shows changes in body weight were expressed as a percentage of body weight measured at 7 weeks of age. One-way ANOVA and Bonferroni's test found that the change in body weight was significantly smaller in CaM-HD than in Vec-HD (p<0.05) starting from week 12, and there is no significant difference between Scr-HD and Vec-HD. * indicates p<0.05 (CaM-HD vs. Vec-HD). FIG. 15B shows Kaplan-Meier survival curves. The first death was at day 88, 78 and 67 in CaM-HD, Vec-HD and Scr-HD mice, respectively. By log-rank comparison, three groups of HD mice did not differ from each other (p>0.05). (n=9˜14 in each group)

FIGS. 16A-16D show video-based gait analysis on the treadmill. Mice were placed on a treadmill belt moving at a speed of 14 cm/s. Gait data were pooled from all four paws. CaM-HD exhibited a significantly greater stride length (FIG. 16A), and a lower stride frequency (FIG. 16B). There is no significant difference between CaM-HD and Vec-HD or Scr-HD in stance width variability (FIG. 16C) and paw area at peak stance (FIG. 16D).

FIGS. 17A-17B show the expression of CaM-fragment enhanced the locomotor activity in R6/2 mice. Spontaneous locomotor activities of R6/2 and WT control mice were recorded in a force-plate actometer apparatus for 30 min. FIG. 17A shows CaM-HD mice displayed a significantly fewer number of low mobility bouts than the two HD control groups at week 12-13. FIG. 17B shows distance traveled by CaM-HD mice was significantly longer than Scr-HD at week 12-13 and Vec-HD at week 12.

FIG. 18 shows CaM-fragment expression delayed the onset of the rotarod defects in R6/2 mice. Mice were placed on a rotating rod with increasing speed, from 4 rpm to 40 rpm in 300 seconds. The latency to fall off the rotarod within this time period was recorded. Two-way ANOVA with repeated measures followed by Bonferroni test found that CaM-HD mice had significantly longer latency to fall than Scr-HD starting from week 10, and than Vec-HD at week 10-11.

FIGS. 19A-19B shows that TGase-modified htt in R6/2 mice striatum was reduced by CaM-fragment expression. FIG. 19A shows the insoluble fraction from mouse striatal homogenates was dissolved with formic acid. Immunopurification (IP) of proteins containing ε-(γ-glutamyl)lysine bonds was performed using 81D4 mAb prebound to Sepharose beads. Immunopurified proteins were then examined on immunoblots (IB) using an antibody against htt. FIG. 19B shows quantification of immunoblots. Data shown are the mean IOD±S.E.M., and they are normalized to CaM-HD mice.

FIGS. 20A-20B show histological evaluation of neuropathology. FIG. 20A Top and middle show immunofluorescent labeling of striatum in R6/2 mice at 14 weeks of age. Htt-aggregates were labeled with htt antibody MAB5374 (red or gray in gray scale), the nuclei were labeled with DAPI (blue or darker gray in grayscale). The composite images show that the percentage of htt-positive nuclei and the size of nuclear htt-aggregates were decreased in CaM-HD as compared to the control-HD mice. Scale bar=30 μm. Bottom, photomicrographs of GFP protein distribution in the brain of representative AAV-injected animals indicated that the same vector-derived CaM-fragment was expressed in the striatum. Scale bar=1 mm. FIG. 20B Top shows montage images of Nissl-stained brain coronal sections from CaM-HD, Vec-HD and CaM-WT mice at the level when the corpus callosum starts to merge in the middle. Scale bar=1 mm. FIG. 20B Bottom shows high magnification of micrograph of the dorsomedial aspect of the striatum from the sections above. There is marked neuronal atrophy with small angulated neurons in Vec-HD mouse, with relative preservation of neuronal size in CaM-HD mouse. Scale bar=50 μm.

FIGS. 21A-21B show expression of CaM-fragment in mouse striatum did not significantly affect the activity CaM kinase II or TGase. FIG. 21A shows mouse striatal homogenates were used to measure CaM kinase II activity. Two-way ANOVA indicates a significant main effect of EGTA. However, there was no significant main effect of mouse group. The interaction between EGTA and group was also not significant. Bonferroni posttest indicates no significant differences in CaM kinase II activity among the various groups in either the presence or absence of EGTA. FIG. 21B shows the presence of the CaM-fragment did not significantly change the levels of TGase activity in either HD or WT mice. However, a significant elevation of TGase activity in three groups of HD mice was observed as compared with WT mice. When 500 μM GTPγS was added, there is a dramatic reduction in all mouse groups. Two-way ANOVA indicates a significant main effect of GTPγS and mouse group, and there is a significant interaction between GTPγS and group.

### DETAILED DESCRIPTION OF THE PREFERRED EMBODIMENTS

Huntington's disease (HD) is a neurodegenerative disease caused by a mutant huntingtin protein containing an expanded polyglutamine tract. This exanded polyglutamine tract may cause abnormal protein-protein interactions, such as increased association with calmodulin (CaM). It has been demonstrated in HEK293 cells that a peptide containing amino acids 76-121 of CaM (hereinafter “CaM-peptide” which is 45 amino acids) interrupted the interaction between CaM and mutant huntingtin, reduced mutant huntingtin-induced cytotoxicity, and reduced transglutaminase (TG) modified mutant huntingtin. It has been found that adeno-associated virus (AAV) mediated expression of the CaM-peptide in differentiated neuroblastoma SH-SY5Y cells stably expressing an N-terminal fragment of huntingtin containing 148 glutamine repeats significantly decreases the amount of TG-modified huntingtin and attenuates cytotoxicity. Importantly, the effect of the CaM-peptide shows selectivity such that total TG activity is not significantly altered by expression of CaM-peptide nor is activity of another CaM-dependent enzyme, CaM kinase II. In vitro, recombinant exon 1 of huntingtin with 44 glutamines(htt-exon 1-44Q) binds to CaM-agarose; the addition of 10 microM of CaM-peptide significantly decreases the interaction of htt-exon 1-44Q and CaM but not the binding between CaM and calcineurin, another CaM-binding protein. These data show that CaM regulates TG-catalyzed modifications of mutant huntingtin, and that specific and selective disruption of the CaM-huntingtin interaction is potentially a new target for therapeutic intervention in HD.

In one embodiment, the present invention includes a polypeptide fragment of CaM that can be used to compete with endogenous full length CaM for binding to mutant huntingtin, thereby interrupting the interaction of endogenous CaM and mutant huntingtin and decreasing the deleterious effects of TG. CaM was divided into three overlapping fragments, which were expressed in human embryonic kidney 293T (HEK-293T) cells that also expressed N-terminal mutant huntingtin (htt-N63-148Q) and TG2. The effects of expression of these calmodulin fragments on TG-catalyzed modifications of mutant huntingtin were studied, and cytotoxicity associated with mutant huntingtin as also studied. It was found that both the center and c-terminal fragments of CaM had positive effects in inhibiting the interaction of CaM and mutant huntingtin without substantial toxicity. To determine if a smaller overlapping region of calmodulin was as successful as previous larger fragments, a 45 amino acid polypeptide (CaM-peptide) was compared with the larger polypeptide by studying its effects on TG-catalyzed modifications of mutant huntingtin and cytotoxicity. It was found that the 45 amino acid fragment (CaM-peptide) was most successful at inhibiting interactions between CaM and mutant huntingtin with low toxicity.

As described, four calmodulin fragments were developed: first 76 amino acids (CaM-N-term), last 72 amino acids (CaM-C-term), 77 amino acids in the center (CaM-center) and the overlapping region of CaM-C-term and CaM-center (CaM-peptide that has 45 amino acids described herein). CaM-C-term, CaM-center, and CaM-peptide significantly decreased amounts of TG-modified huntingtin by 40-60%, and cytotoxicity decreased up to 40% compared to cells not expressing any calmodulin construct. Carbachol-stimulated release of intracellular calcium is significantly higher in cells expressing N-terminal mutant huntingtin and TG2 compared to vector-transfected cells; expression of either CaM-center or CaM-peptide in these cells returned the levels of carbachol-stimulated intracellular calcium release to control values. Furthermore, CaM-peptide expression significantly decreased huntingtin binding to calmodulin. These data further suggest that calmodulin regulates TG2 activity, plays a role in the disease-related modifications to mutant huntingtin and that disruption of calmodulin-mutant huntingtin interaction is potentially a new target for therapeutic intervention in HD.

Accordingly, the present invention includes a polypeptide that is a fragment of CaM, which can be used as a treatment of Huntington's disease. Testing in cells demonstrates that this polypeptide is effective at inhibiting the interaction between CaM and mutant huntingtin, and has reduced (low) cytotoxicity measured using an LDH assay. In neuronal cells, the polypeptide has shown specificity toward mutant huntingtin, and reduced cell toxicity. In HD transgenic mice, the polypeptide can reduce motor dysfunction, and reduces weight loss.

The approach taken with the present invention is to use a polypeptide to inhibit the interactions of both TG and CaM with mutant huntingtin protein. The polypeptide specifically inhibits TG from being able to modify mutant huntingtin while leaving TG and CaM perfectly intact to interact with other normal proteins in the brain and the rest of the body. As such, other functions of TG and CaM are maintained so that the therapy is specific without any unwanted off-targeted interactions and without adverse side effects.

The polypeptide can be a portion of CaM. The polypeptide can include 45 amino acids and having the sequence KDTDSEEEIREAFRVFDKDGNGYISAAELRHVMTNLGEKLTDEEV (SEQ ID NO: 1), which is referred to herein as the CaM-peptide. The CaM-peptide of the present invention interrupts the interaction of CaM with mutant huntingtin protein. Additionally, the polypeptide leaves CaM available to modify other enzymes that it normally interacts with and regulates. As such, other functions of CaM are maintained, with further specifies the therapy with limited unwanted off-targeted interactions and without adverse side effects.

The CaM-peptide directly inhibits the binding of mutant huntingtin from CaM. It is surprising and unexpected that the binding between CaM-peptide and huntingtin is unique and that the sequence for binding to huntingtin is a unique peptide sequence. The CaM protein does not interact with normal huntingtin protein, and it appears that the binding site between mutant huntingtin and CaM is unique. We have not been able to find another binding protein that interacts with the same sequence of CaM.

Accordingly, the CaM-peptide and the larger polypeptides described herein that contain the CaM-peptide sequence show that it is feasible to have a polypeptide that binds with mutant huntingtin to as to prevent mutant huntingtin to bind with CaM or other polypeptides or proteins. As such, the present invention can generally relate to the approach of using a polypeptide or drug to bind with mutant huntingtin so as to disrupt the interaction between mutant huntingtin and CaM or other proteins.

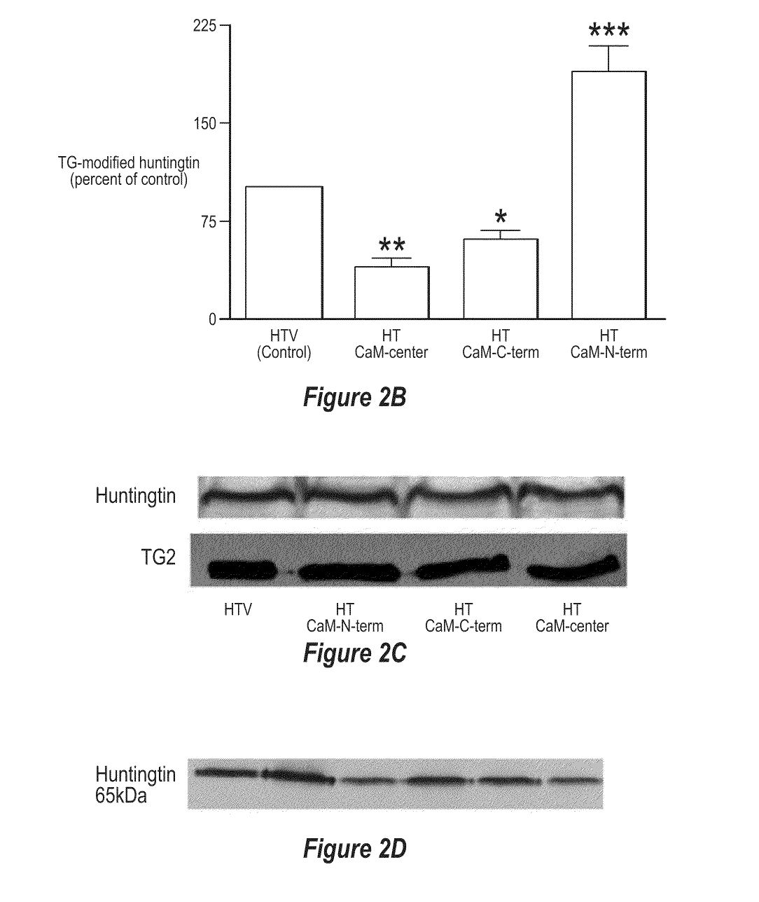

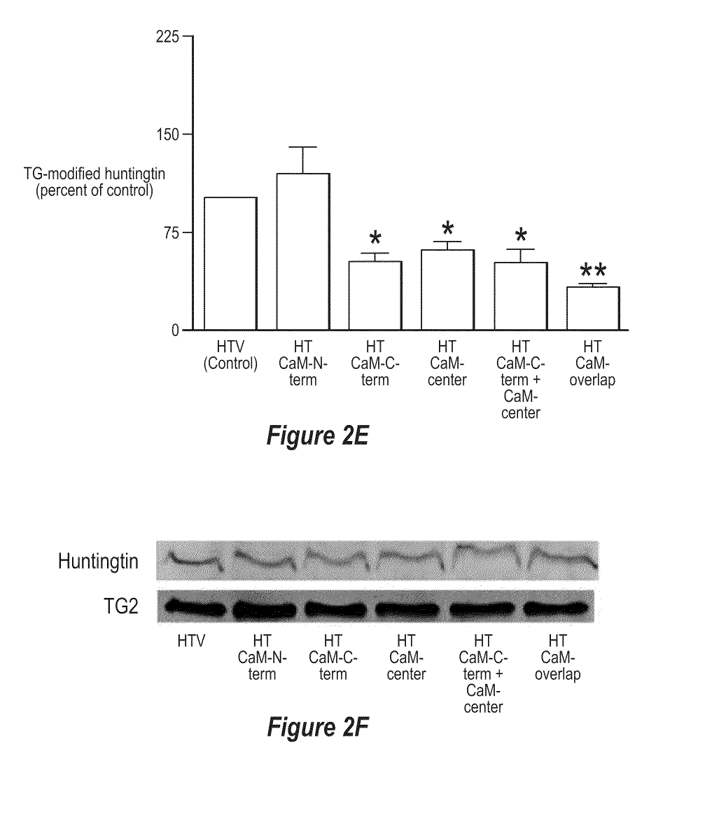

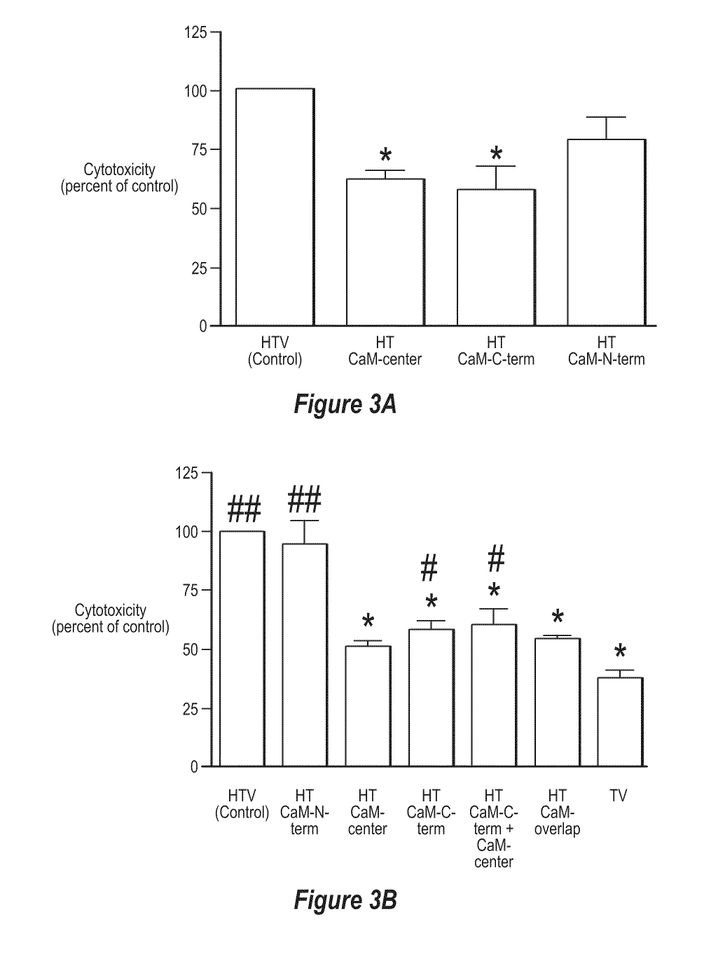

The results presented here show that cytotoxicity and TG-catalyzed modifications of mutant huntingtin were attenuated in cells expressing CaM-center or CaM-C-term polypeptides, individually (FIGS. 2A-2B and FIG. 3A). Since CaM-center and CaM-C-term produced similar results two constructs were further studied by examining their effects on intracellular Ca²⁺ regulation. The similarities between those two peptides that worked well were used to identify the region that overlapped between the two of them, and later testing identified the 45 amino acid polypeptide (CaM-peptide) binds with mutant huntingtin and inhibits binding with CaM.

Accordingly, the 45 amino acid sequence of CaM-peptide contains within it the sequence for disrupting the binding of mutant huntingtin and calmodulin. Accordingly, truncation studies can be conducted to identify the smallest number of amino acids in sequence that perform the function of binding with mutant huntingtin so as to block interactions with CaM or other proteins. As such, the CaM-peptide can be further truncated at the C-terminus or N-terminus by up to about 5 amino acids, about 10 amino acids, or even 15 amino acids so long as the functionality of inhibiting the interaction between native CaM and mutant huntingtin is maintained. It has now been conceived that a portion or subsection of the CaM-peptide may also be effective in binding to mutant huntingtin to inhibiting binding with CaM. Accordingly, we have conceived of conducting molecular modeling and identifying the 3-D structures, and then making smaller peptides based on the 3-D structure that also bind mutant huntingtin to inhibit binding with CaM. It is thought that truncating the 45 amino acid CaM-peptide can result in a smaller polypeptide with substantially the same functionality. For example, the CaM-peptide may be truncated by about 5, 10, or 15 or more amino acids from the N-terminus and/or C-terminus may result in a smaller polypeptide that functions similarly to the CaM-peptide as described herein.

In one embodiment, the present invention is a polypeptide containing the sequence KDTDSEEEIREAFRVFDKDGNGYISAAELRHVMTNLGEKLTDEEV (SEQ ID NO: 1) (CaM-peptide). The polypeptide can be purified, recombinant, engineered, synthesized, or otherwise artificial. As used herein, an artificial polypeptide is a polypeptide that is not a full, natural protein, and thereby an artificial polypeptide is man-made or from a man-made process. The polypeptide containing the sequence is not CaM or full, natural protein or polypeptide of CaM.

In one embodiment, the present invention is a portion of the CaM-peptide. The portion can have a sequence of EEEIREAFRVFDKDGNGYISAAELRHVMTNLGEKLTDEEV (SEQ ID NO: 2); KDTDSEEEIREAFRVFDKDGNGYISAAELRHVMTNLGEKL (SEQ ID NO: 3); EEEIREAFRVFDKDGNGYISAAELRHVMTNLGEKL (SEQ ID NO: 4); EAFRVFDKDGNGYISAAELRHVMTNLGEKLTDEEV (SEQ ID NO: 5); KDTDSEEEIREAFRVFDKDGNGYISAAELRHVMTN (SEQ ID NO: 6); EEEIREAFRVFDKDGNGYISAAELRHVMTN (SEQ ID NO: 7); EAFRVFDKDGNGYISAAELRHVMTNLGEKL (SEQ ID NO: 8); FDKDGNGYISAAELRHVMTNLGEKLTDEEV (SEQ ID NO: 9); KDTDSEEEIREAFRVFDKDGNGYISAAELR (SEQ ID NO: 10); EEEIREAFRVFDKDGNGYISAAELR (SEQ ID NO: 11); FDKDGNGYISAAELRHVMTNLGEKL (SEQ ID NO: 12); EAFRVFDKDGNGYISAAELR (SEQ ID NO: 13); FDKDGNGYISAAELRHVMTN (SEQ ID NO: 14); FDKDGNGYISAAELR (SEQ ID NO: 15); or an analog or derivative thereof. Also, the polypeptide of the present invention can be one of the recited polypeptides that is truncated at the C-terminus or N-terminus by up to about 1, 2, 3, 4, or 5 amino acids, about 6, 7, 8, 9, or 10 amino acids, or even 11, 12, 13, 14, or 15 amino acids so long as the functionality of inhibiting the interaction between native CaM and mutant huntingtin is maintained.

In one embodiment, the polypeptide of the present invention is an analog or derivative of one of the polypeptides described herein so long as the functionality of inhibiting the interaction between native CaM and mutant huntingtin is maintained. As used herein, an “analog” of a polypeptide can include amino acid substitutions at one or more amino acid positions so long as the functionality of inhibiting the interaction between native CaM and mutant huntingtin is maintained. For example, analogs of the polypeptides can include nonpolar or hydrophobic amino acids being substituted with other nonpolar or hydrophobic amino acids (G, A, V, L, I, M, F, W, P); polar or hydrophilic amino acids being substituted with other polar or hydrophilic amino acids (S, T, C U, N Q); or electrically charged and negative and hydrophilic (D, E) being substituted with the same type of amino acids; and electrically charged and positive and hydrophilic being substituted with the same type of amino acids (K, R, H). As used herein the derivatives of the polypeptides of the invention can include single or multiple acid or functional group substitutions, where for example, hydrogen can be substituted with halogens, C1-C4 alkyl groups that are straight or branched or aromatic rings (one or more) that contain carbon or hetero atoms. Also, traditional amino acid derivatives can be included in the polypeptides of the present invention, examples of which include D-isomers, mono and di-hydrochlorides, methyl esters, Fmoc or Boc-blocked, hydroxides, or the like.

In one embodiment, the present invention can include a method of inhibiting calmodulin from interacting with a mutant huntingtin protein. Such a method can include: providing a mutant huntingtin protein having a polyglutamine sequence in the presence of a calmodulin protein; and contacting the mutant huntingtin protein with an artificial polypeptide, said polypeptide sequence being capable of interacting with the mutant huntingtin so as to inhibit interactions between the mutant huntingtin and calmodulin proteins. Examples of artificial polypeptides are provided herein. Additionally, the polypeptide binds to the mutant huntingtin protein and inhibits the mutant huntingtin protein from interacting with a transglutaminase. The mutant huntingtin protein can be located within a cell, such as a neuronal cell. Also, the mutant huntingtin protein can be located within a subject having Huntington's Disease or susceptible thereto.

A subject having Huntington's disease can be diagnosed as is routine in the art. Also, a subject susceptible to Huntington's disease can be diagnosed as having the mutant huntingtin gene. A subject receiving the treatment of the present invention can have the symptoms of Huntington's disease reduced, alleviated, or otherwise inhibited. This can include improvements in coordination and increased steady gait, improved coordination, lessened jerky body movements, increased mental abilities, increased behavior and psychiatric conditions. Also, the treatment described herein can lessen any of these symptoms or decrease the onset thereof. The treatment can be provided as a prophylactic to inhibit these symptoms from developing and/or advancing.

Synthesis of peptides as described herein is well established. As such, with the disclosure of the amino acids of the polypeptides, a chemist can prepare the cyclic tetrapeptides of the present invention through routine experimentation. Aditionally, biological techniques can be used so that an organism, such as a bacteria, can be transformed with a gene encoding for the production of the polypeptides, and such polypeptides can then be isolated for use.

The polypeptides can be administered to a subject via traditional routes of administration. Such routes include intravenous, intraperitoneal, subcutaneous, intrathecal, inhalation, nasal, transdermal, and the like. For example, subcutanteous (s.c.) administration of the polypeptides may result in an effecitve amount to cross the BBB so as to be active in the brain. Otherwise, the polypeptides can be administered directly into the brain. As such, the polypeptides can be included in a composition with a pharmaceutically acceptable carrier that is selected based on the mode of administration.

In one embodiment, the polypeptides can be encapsulated into a microsphere for oral administration. Alternatively, the polypeptides can be formulated so as to allow passage through and absorption from the gastrointestinal tract.

However, there are other possible routes of administration that minimize or eliminate the need for regular injection of the compound. For example, depot formulations, such as temperature-reversable polymers or hydrogels, where the duration of action (e.g., from days to months) can be regulated by the formulation. Also, inhaled formulations and transdermal formulations can be prepared. The preparation of drug delivery formulations that include the proper adjuvants are well known for specific modes of delivery. Depot formulations for injection can be developed that control the duration of action (from a few days to months) minimizing the frequency of injection. Alternatively a formulation for inhalation (similar to the recently introduced inhaled insulin product) can be developed so that the drug does not have to be injected. Also, nasal administration can also be effective.

One mode of administration can be via a gene delivery system that delivers a nucleic acid encoding the polypeptide so that the polypeptide is produced within a cell. The gene can be carried with a viral gene carrier or a non-viral gene carrier. Viral gene carriers can include retrovirus, lentivirus, adenovirus, adeno-associated virus, or other viral gene carriers. A non-viral gene carrier can be included in complexes with cationic liposomes and cationic polymers. Genes can be prepared for production of the polypeptide by identification of the polypeptide sequence and using well-known techniques in genetic engineering. The genes can then be included within a viral particle such that the polypeptide is produced upon the viral particle entering a cell. Examples of adeno-associated viruses containing genes encoding embodiments of the polypeptides are described in the examples.

In one embodiment, the present invention can include methods for screening substances (e.g., small polypeptides or molecules) to find inhibitors that inhibit CaM from binding with mutant huntingtin. The screening methods can be performed substantially as described herein with a small molecule or polypeptide being screened for activity as an inhibitor. The inhibitors can have a bioactivity similar to the activity shown for the CaM-peptide. It is thought that small polypeptides that are less than 20 amino acids, or less than 15, or less than 10, and preferably less than 5 amino acids may be effective at inhibiting the binding between CaM and mutant huntingtin.

In one embodiment, the screening can be conducted to determine a smaller polypeptide that can function as an inhibitor, as well as determine the minimal sized polypeptide needed to interrupt the interaction between mutant huntingtin and CaM. The smaller polypeptide can have more favorable delivery properties.

Additionally, a small polypeptide inhibitor can be used in modeling studies to map the interactions between the polypeptide and with mutant huntingtin and/or CaM. Such modeling can be used to design or identify features to be included within a molecule inhibitor, which may be a pharmacophore/peptidomimetics for the treatment of Huntington's disease. The modeled inhibitors (e.g., peptidomimetics) can be screened for their ability to disrupt the binding of mutant huntingtin fragments to CaM substantially as described herein. The smaller peptides, small molecules, and/or pharmacophore/peptidomimetics can be considered both novel drug target and a novel drug for use as described herein. Additionally, any small polypeptide identified by the screens can be prepared into a viral delivery vehicle as described herein.

The screening can be initiated by preparing plasmid vectors to express smaller polypeptide constructs (e.g., from CaM). For example, the 45mer CaM-peptide in the pEF6 vector can be used as a template, and the appropriate primers can then be used for PCR amplification. This is similar to the approach used to prepare and express CaM-peptide (Dudek et al, 2008). The amplified DNA can be cloned into the pEF6 vector and the sequence will be verified.

Smaller polypeptides and compounds which are mimics of these smaller peptides (i.e., pharmacophore/peptidomimetics) can be screened for their ability to disrupt the binding of calmodulin to mutant huntingtin (including N-terminal fragments of mutant huntingtin). Binding of mutant huntingtin to calmodulin can be assessed using calmodulin-agarose (Sigma, St. Louis, Mo.) as we previously described (Dudek et al, 2008). HEK-293 cells can be transfected with a 63 amino acid N-terminal huntingtin fragment containing 148 polyglutamine repeats (htt-N63-148Q), transglutaminase 2 and either vector or a test construct (e.g., CaM construct), and forty-eight hours later, cells can be harvested and resuspened in lysis buffer. Then cell lysates can be mixed with calmodulin-agarose in 20 mM Tris-HCl buffer (pH 7.5) containing 1 mM CaCl₂, 100 mM NaCl and 1 mM MgCl₂ and incubated overnight at 4° C. After washing four times with the same buffer, the agarose-bound proteins can be eluted with 20 mM Tris-HCl buffer (pH 7.5) containing 10 mM EGTA along with Laemmli sample loading buffer. The fractions obtained can be analyzed by SDS-gel electrophoresis followed by immunoblotting. Small polypeptides (e.g., CaM constructs) that inhibit the binding of mutant huntingtin to calmodulin can be further tested for protection against the cytotoxicity induced by expression of the mutant huntingtin protein and for inhibition of transglutaminase-modifications to mutant huntingtin protein using immunoprecipitation and immunoblotting.

The screening experiments can produce data on the efficacy of the smaller polypeptides for protective effects in cell culture models. Any peptide can be included in a vector for delivery and expression of the smaller polypeptides. Then, a viral vector can be produced for the delivery of genes to express the small polypeptides. The viral vectors can then be used to screen the smaller polypeptides in the R6/2 transgenic mouse model of Huntington's disease to determine if the small polypeptides function as inhibitors in vivo. Additionally, the small molecules or other inhibitors identified to inhibit the interaction between CaM and mutant huntingtin can be screened with the R6/2 transgenic mouse.

The strategy to interrupt the interaction between mutant huntingtin and CaM is a novel approach for the treatment of Huntington's disease and has not been explored before now. The strategy can be implemented with polypeptides (e.g., CaM-peptide) as described herein as well as smaller polypeptides and small molecules or traditional drugs.

### Definitions

As used herein, the terms “an effective amount”, “therapeutic effective amount”, or “therapeutically effective amount” shall mean an amount or concentration of a compound according to the present invention which is effective within the context of its administration or use. Thus, the term “effective amount” describes concentrations or amounts of the polypeptides according to the present invention which may be used to produce a favorable change in the disease or condition treated, inhibited, or prevented. The polypeptides of the present invention can be administered in a therapeutically effective amount to treat, inhibit, and/or prevent Huntington's disease or symptom thereof. An effective amount of the polypeptides can be used for binding with mutant huntingtin protein to inhibit interactions with calmodulin.

As used herein, the term “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient. The compositions of the present invention can include a pharmaceutically acceptable excipient.

As used herein, the term “pharmaceutically acceptable carrier” means a drug carrier that is useful in preparing a pharmaceutical composition that is generally safe, non-toxic and neither biologically nor otherwise undesirable, and includes a carrier that is acceptable for veterinary use as well as human pharmaceutical use. The compositions of the present invention can include a pharmaceutically acceptable carrier.

As used herein, the term “treating” or “treatment” of a disease, including Huntington's disease, includes: (a) preventing the disease, i.e. causing the clinical symptoms of the disease not to develop in a mammal that may be exposed to or predisposed to the disease but does not yet experience or display symptoms of the disease; (b) inhibiting the disease, i.e., arresting or reducing the development of the disease or its clinical symptoms; or (c) relieving the disease, i.e., causing regression of the disease or its clinical symptoms.

As used herein, a “subject” or a “patient” refers to any mammal (preferably, a human), and preferably a mammal that may have or be susceptible to Huntington's disease or have mutant huntingtin protein. Examples of a subject or patient include a human, a non-human primate, a cow, a horse, a pig, a sheep, a goat, a dog, a cat or a rodent such as a mouse, a rat, a hamster, or a guinea pig. Generally, the invention is directed toward use with humans.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference in their entirety.

### Experimental

### Example Set A Protocols

Calmodulin fragment constructs. The pEYFP-C1 plasmid expressing wild-type full length calmodulin (generous gift from Dr. John Raymond, Medical University of South Carolina) was used as a template for the generation of the CaM fragment constructs. PCR primers used for generating these plasmids are as follows:

Primer 1:

(SEQ ID NO: 16)

AGGGCTAGGTACCGAATCATGGCTGATCAGCGTACC
(Forward: CaM N-term)
Primer 2:

(SEQ ID NO: 17)

CCCTCTAGACTCGAGCGGCCGCCACATTTTTCTAGCCATC
(Reverse: CaM N-term)
Primer 3:

(SEQ ID NO: 18)

AGGGCTAGGTACCGAATCATGAAGGACACAGACAGTGAGGAGGAGATC
(Forward: CaM C-term and Overlap)
Primer 4:

(SEQ ID NO: 19)

CCCTCTAGACTCGAGCGGCCGCCATTTTGCAGTCATCATCTG
(Reverse: CaM C-term)
Primer 5:

(SEQ ID NO: 20)

AGGGCTAGGTACCGAATCATGGAAGCAGAGCTGCAGGATATG
(Forward: Center Region)
Primer 6:

(SEQ ID NO: 21)

CCCTCTAGACTCGAGCGGCCGCCACACCTCATCGGTCAG
(Reverse: CaM Center Region and Overlap)

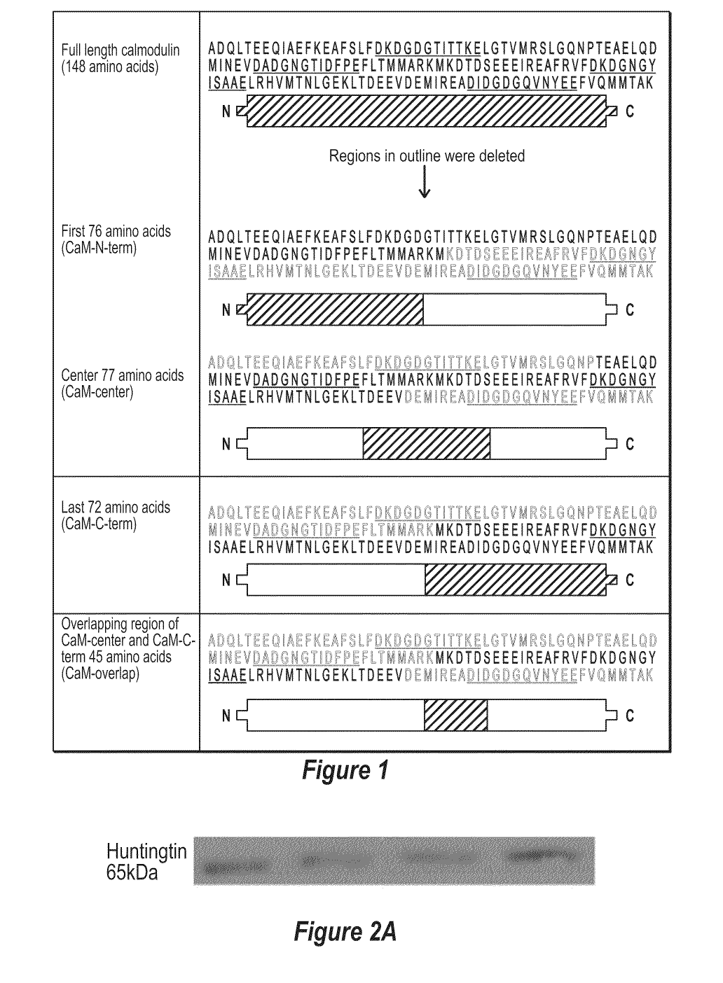

CaM has four Ca²⁺ binding sites, and the first three CaM constructs were designed to include at least two of these sites (FIG. 1). The CaM N-term construct contains the first two Ca²⁺ binding sites, and was generated by amplification of the first 231 bp of wild-type calmodulin using forward and reverse primers 1 and 2 containing KpnI and NotI restriction sites. The CaM C-term construct contains the third and fourth Ca²⁺ binding sites, and was generated by amplification of the last 216 bp of wild-type calmodulin using forward and reverse primers 3 and 4 containing KpnI and NotI restriction sites. The CaM center-region construct contains the second and third Ca²⁺ binding sites, and was generated by amplification of the center 231 bp of wild-type calmodulin using forward and reverse primers 5 and 6 containing KpnI and NotI restriction sites. The CaM overlap construct is the overlapping region of CaM-center and CaM C-term (FIG. 1) and therefore only contains the third Ca²⁺ binding site. CaM overlap was generated by amplification of the overlapping 135 bp of the C-term and center region constructs, using forward and reverse primers 3 and 6 containing KpnI and NotI restriction sites. The resultant PCR products were digested and cloned into the KpnI and NotI sites in the pEF6 vector (generous gift from Dr. Vinay Kumar, University of Chicago). Constructs were verified by sequencing.

Antibodies. A mouse anti-myc monoclonal antibody directed against the amino acid sequence EQKLISEEDL (SEQ ID NO: 22) was used at a 1:1,000 for immunoblots and 1:500 for immunocytochemistry (Invitrogen, Carlsbad, Calif.). The antibody to transglutaminase 2, TG-100, was used at a dilution of 1:1,000 for immunoblots (NeoMarkers, Union City, Calif.).

Cell culture system. HEK-293T cells were grown in OptiMEM reduced serum medium (Invitrogen, Carlsbad, Calif.) containing nonessential amino acids, antimycotic and antibiotic agents, and 10% FBS, in the presence of 5% CO₂ at 37° C. Cells were grown to 80-90% confluency and transfected using Lipofectamine Plus (Invitrogen, Carlsbald, Calif.). Transfection of the following constructs was performed in various combinations: vector (V), N-terminal huntingtin fragment containing 148 polyglutamine repeats (htt-N63-148Q), transglutaminase 2 (TG2), N-terminal CaM construct (CaM-N-term), C-terminal CaM construct (CaM-C-term), center region CaM construct (CaM-center), and overlap CaM construct (CaM-overlap). Forty-eight hours after transfection, cells were harvested and resuspened in lysis buffer containing 50 mM Tris-HCl, pH 8.8, 100 mM NaCl, 5 mM MgCl₂, 0.05% NP-40, 1 mM EDTA (Tang T S, Tu H, Chan E Y, et al. Huntingtin and huntingtin-associated protein 1 influence neuronal calcium signaling mediated by inositol-(1,4,5)triphosphate receptor type 1. Neuron 2003;39:227-239) and 1:1,000 protease inhibitor mixture (Sigma, St. Louis, Mo.). Insoluble fractions were prepared by centrifuging the cell lysates at 12,000×g for 5 minutes, the supernatant was then removed and the pellet was resuspended in 95% formic acid and incubated at 37° C. for 40 minutes. The formic acid was then removed under vacuum. The pellets were then resuspended in 10mM Tris-HCl, pH7.5, 0.14M NaCl, and 0.1% Tween 20, for further analysis.

Immunoprecipitation. Immunopurification of proteins containing transglutaminase-catalyzed ε-(γ-glutamyl)lysine bonds was performed using 81D4 Mab prebound to Sepharose beads using a protocol developed by CovalAb (Lyon, France) as previously described (Young A B. Huntingtin in health and disease. J Clin Invest 2003;111:299-302.). The eluted immunopurified TG-modified proteins were stored at −80° C. until immunoblot analysis.

Immunoblots. Proteins were separated on 12% SDS- polyacrylamide gels then electrophoretically transferred to nitrocellulose membranes. Membranes were blocked in 5% nonfat dried milk in Tris-buffered saline with 0.1% Tween 20. After overnight incubation in primary antibody, membranes were incubated in goat anti-mouse secondary antibody conjugated to horseradish peroxidase (Jackson ImmunoResearch, West Grove, Pa.). Signal was detected using enhanced chemiluminescence (ECL) Western blotting detection reagents (Amersham Biosciences, Piscataway, N.J.). Immunoblots were quantified by calculating the sum of the densities of all of the pixels within each protein band as the integrated optical density (IOD), using Scion Image for Windows (Scion, Frederick, Md.). Film background IOD was measured and subtracted from each band IOD. Measurements were done in triplicate and the mean was calculated.

Cytotoxicity Assay. Transfections were performed and 96 hours post transfection, lactate dehydrogenase (LDH) assays were performed using the CytoTox 96® Non-Radioactive Cytotoxicity Assay (Promega, Madison, Wis.). Briefly, half the media from the cells was removed and placed in the wells of a 96-well plate. The cells were frozen in the remaining media at −80° C. followed by thawing in order to lyse the cells. Cells and media were briefly centrifuged and the media was removed and placed in the remaining wells of the 96 well plate. Substrate solution was added to all samples and incubated at room temperature in the dark for 30 minutes. Stop solution was added and the absorbance was recorded at 490 nm. Percent cytotoxicity was determined by the LDH in media before lysing cells/LDH in media after cells were lysed.

Ca²⁺ Measurement. Transfections were performed as described above and 96 hours post transfection the cells were incubated in modified Krebs buffer containing 5 μM Fura-2 AM (Molecular Probes, Carlsbad, Calif.), 0.1% bovine serum albumin, and 0.02% Pluronic F127 detergent for 120 min at room temperature in the dark. The cells were then washed and incubated in Krebs buffer for 30 minutes in the dark. The buffer was removed and fresh Krebs buffer was placed on the cells. Cells were treated with 60 μM carbachol and Fura-2 fluorescence ratio was determined by measuring fluorescence at 380 nm/510 nm and 340 nm/510 nm.

Immunocytochemistry. Cells were transfected with myc-tagged CaM constructs and plated onto collagen-coated chamber slides. 48 hours post-transfection cells were washed in PBS and then fixed in 4% paraformaldehyde for 20 minutes at room temperature then washed again in PBS. The cells were permeabilized in PBS with 0.15% Triton for 15 minutes at room temperature and non-specific binding was blocked by incubating the cells in 5% normal goat serum (NGS) and 0.15% Triton. Cells were incubated overnight in anti-myc antibody, washed, then incubated in goat anti-mouse antibody conjugated to rhodamine red (Jackson ImmunoResearch, West Grove, Pa.). Coverslips were mounted with media containing DAPI.

Fluorescence microscopy. Cells were examined using an Olympus (Tokyo, Japan) fluorescent inverted research microscope configured with Image-Pro Plus 4.5 software (VayTek, Fairfield, Iowa). Monochrome images were captured using Retiga EX 1350 camera and Volume Scan 3.1 software (VayTek) and then were pseudocolored and merged using Image-Pro Plus 4.5 software.

Calmodulin binding. Binding of huntingtin to calmodulin was studied using calmodulin-agarose (Sigma, St. Louis, Mo.). Cells were transfected with htt-N63-148Q, TG2 and either vector or overlap CaM construct and harvested as described above. Then cell lysates were mixed with calmodulin-agarose in 20 mM Tris-HCl buffer (pH 7.5) containing 1 mM CaCl₂, 100 mM NaCl and 1 mM MgCl₂ and incubated overnight at 4° C. After washing four times with the same buffer, the agarose-bound proteins were eluted with 20 mM Tris-HCl buffer (pH 7.5) containing 10 mM EGTA along with Laemmli sample loading buffer. The fractions obtained were analyzed by SDS-gel electrophoresis followed by immunoblotting.

### Example Set A Results

It has been found that polypeptides of the C-terminus and center region of calmodulin decrease TG-catalyzed modifications of mutant huntingtin. To determine if there was a region of CaM that was able to decrease TG-catalyzed modifications of mutant huntingtin an N-terminal fragment of CaM (CaM-N-term), a center fragment of CaM (CaM-center) and a C-terminal fragment of CaM (CaM-C-term) were created (FIG. 1). HEK-293T cells were transfected with htt-N63-148Q, TG 2 and one of the following: vector, CaM-N-term, CaM-C-term, or CaM-center. Cells expressing htt-N63-148Q, TG2 along with CaM-C-term or CaM-center show almost a 2-fold decrease in TG-modified huntingtin compared to cells expressing htt-N63-148Q, TG2 and vector (control) (FIGS. 2A and 2B). However, cells expressing htt-N63-148Q, TG2, and CaM-N-term show no significant decrease in TG-modified huntingtin, but rather an increase compared to control cells (FIGS. 2A and 2B). Although a large amount of the TG-modified mutant huntingtin is the monomeric form, upon longer exposure of the blot to x-ray film, we were able to detect a mobility shift which indicates higher molecular weight forms of the TG-modified huntingtin (data not shown). Furthermore, there was equal expression of total huntingtin and TG2 in all transfectants suggesting that differences in huntingtin or TG2 protein does not account for the observed differences in TG-catalyzed modifications to mutant huntingtin (FIG. 2C).

It has been found that polypeptides having a sequence of the C-terminus and center region of calmodulin decrease mutant huntingtin associated cytotoxicity. HEK-293T cells were transfected as described above and 96 hours post transfection cells cytotoxicity was measured to further examine the effects of the CaM fragments. Cells expressing the CaM-C-term and CaM-center constructs showed a significant decrease in cytotoxicity by approximately 40% compared to cells expressing htt-N63-148Q, TG2 and no CaM construct (FIG. 3A). Although there was a slight decrease in cytotoxicity in cells expressing CaM-N-term, it was not significantly different from htt-N63-148Q, TG2 and vector expressing control cells (FIG. 3A). The procedure for the cytotoxicity assay prevents analysis of protein levels. However, transfections for the cytotoxicity assays were performed in parallel with TG-modified huntingtin assays, so the equal levels of expression of mutant huntingtin and TG 2 observed in the TG-modification assays (FIG. 2C) indicate equal protein levels in the cytotoxicity assays also.

The effects of CaM-overlap (which is also CaM-peptide) and combined CaM-center and CaM-C-term on TG-catalyzed modifications of huntingtin, mutant huntingtin associated cytotoxicity were studied. Since both the CaM-C-term and CaM-center constructs led to significantly decreased TG-catalyzed modifications of huntingtin, significantly reduced cytotoxicity, the common region of CaM shared by these two fragments was developed and termed CaM-overlap or CaM-peptide (FIG. 1). To determine if CaM-overlap would be as successful as the previous fragments we compared its effects on TG-catalyzed modifications of mutant huntingtin and cytotoxicity to the effects of the previous fragments as well as co-expression of CaM-C-term and CaM-center. HEK-293T cells were transfected with htt-N63-148Q, TG 2 and one of the following constructs: vector, CaM-overlap, CaM-N-term, CaM-C-term, CaM-center or the combination of CaM-C-term and CaM-center. Cells expressing htt-N63-148Q, TG2 and vector result in TG-catalyzed modifications of huntingtin (FIGS. 2D and E). Similar to that found in previous experiments, expression of CaM-center as well as expression of CaM-C-term result in almost a 2-fold decrease in TG-catalyzed modifications of huntingtin, but CaM-N-term did not reduce the TG-catalyzed modifications of huntingtin (FIGS. 2D and 2E). Expression of CaM-C-term along with CaM-center also results in a 2-fold decrease in the amount of TG-modified huntingtin, therefore expression of CaM-C-term and CaM-center together does not result in either an additive or synergistic effect. In cells expressing htt-N63-148Q, TG2 and CaM-overlap, the amount of TG-modified huntingtin protein decreased approximately 3-fold (FIGS. 2D and 2E). Furthermore, there was equal expression of total huntingtin and TG 2 in all transfectants suggesting that differences in huntingtin or TG 2 protein does not account for the observed differences in TG-catalyzed modifications to mutant huntingtin (FIG. 2F).

Next, we determined if expression of CaM-overlap and/or co-expression of CaM-center and CaM-C-term would reduce cytotoxicity compared to cells not expressing any CaM construct, and if this reduction would be greater than the reduction that occurs with expression of the CaM-C-term or CaM-center alone. Also, in order to determine if expression of the CaM constructs would reduce huntingtin-associated cytotoxicity back to control levels we compared cytotoxicity in cells expressing the CaM constructs and htt-N63-148Q to that in cells not expressing htt-N63-148Q. The transfections were performed and 96 hours post transfection cells cytotoxicity was measured. Cytotoxicity was approximately two-times higher in cells expressing htt-N63-148Q, and TG2 compared to cells expressing TG2 but not htt-N63-148Q (FIG. 3B). As before, expression of CaM-N-term had no significant effect on cytotoxicity but expression of CaM-C-term and CaM-center reduced cytotoxicity by about 40% and 50% respectively, compared to cells expressing htt-N63-148Q and TG2 (FIG. 3B). Similarly co-expression of CaM-C-term along with CaM-center and expression of CaM-overlap also lead to 40% and 50% decreases in cytotoxicity, respectively (FIG. 3B). Therefore, co-expression of the two constructs or expression of the overlap construct did not lead to any further decrease in cytotoxicity. However, the CaM-center and CaM-overlap constructs were the only constructs which reduced cytotoxicity to levels that are not significantly different from those in cells lacking htt-N63-148Q (FIG. 3B). As previously, the cytotoxicity assays were performed in parallel with TG-modified huntingtin assays, so the equal levels of expression of mutant huntingtin and TG2 observed in the TG-modification assays (FIG. 2F) indicate equal protein levels in the cytotoxicity assays also.

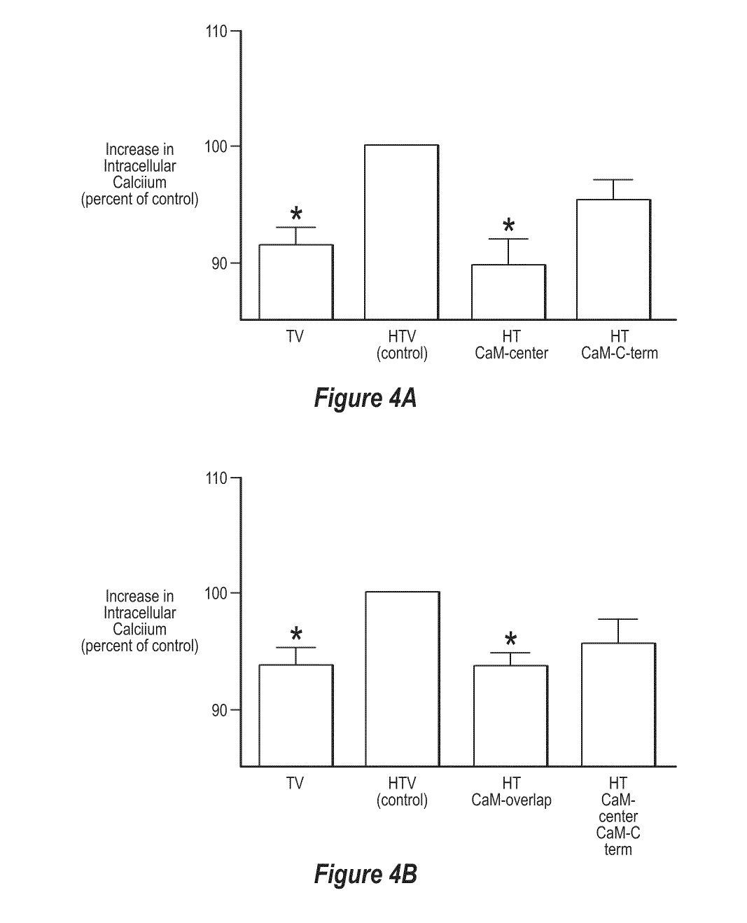

It was found that a polypeptide having a sequence of the center region of CaM attenuates mutant huntingtin associated intracellular Ca²⁺ disturbances. Since expression of CaM-center and CaM-C-term had equivalent results for levels of TG-modified mutant huntingtin and cytotoxicity these constructs were further compared by examining their effects on intracellular Ca²⁺ release. HEK-293T cells were transfected as before, then IP₃R-mediated Ca²⁺ release was stimulated using the muscarinic receptor agonist carbachol and the released Ca²⁺ was measured. Ca²⁺ release was increased by approximately 10% in cells expressing htt-N63-148Q and TG2 compared to non-htt-N63-148Q expressing cells (FIG. 4A). In cells expressing htt-N63-148Q, TG2 and CaM-center, Ca²⁺ release was significantly decreased by 10% compared to htt-N63-148Q and TG2 expressing cells. In cells over-expressing the CaM-C-term in htt-N63-148Q and TG2, there was a small decrease that did not reach statistical significance (FIG. 4A).

Since the effects of CaM-overlap on TG-catalyzed modifications of mutant huntingtin and mutant huntingtin associated cytotoxicity were more efficient than the effects of CaM-center or CaM-C-term, CaM-overlap will be the fragment used in future animal studies. Therefore, we wanted to further investigated CaM-overlap by examining its effects on intracellular Ca²⁺ regulation. Again in cells expressing htt-N63-148Q and TG2, release of Ca²⁺ increased compared to cells which do not express htt-N63-148Q. In cells co-expressing CaM-center along with CaM-C-term, there was a decreased release of Ca²⁺ compared to htt-N63-148Q and TG2 expressing cells, which did not reach significance. However, expression of CaM-overlap reduced the amount of Ca²⁺ released back to levels released by cells not expressing htt-N63-148Q (FIG. 4B).

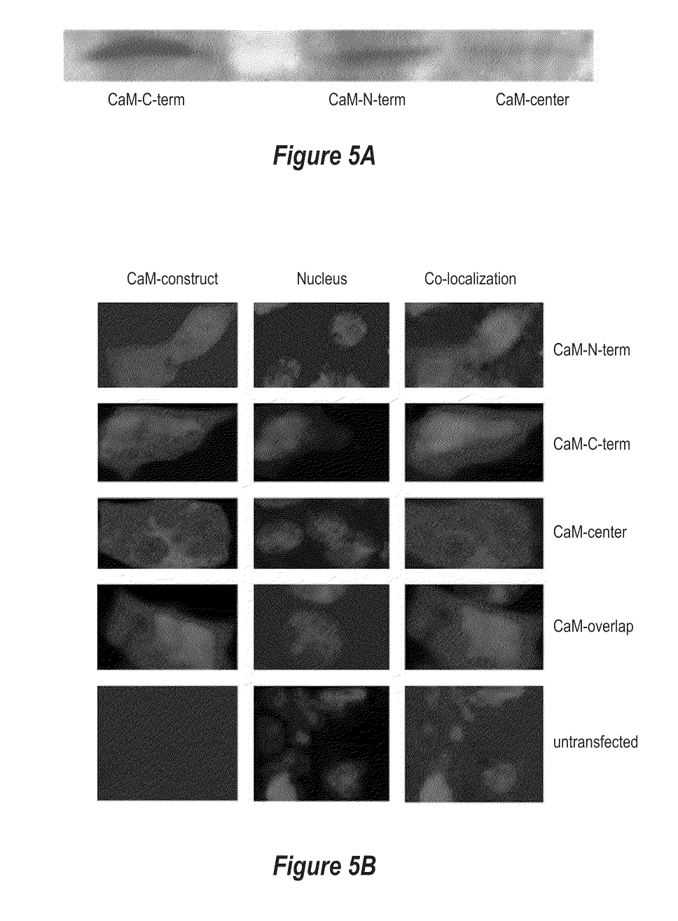

The localization and expression of CaM constructs was studied. We wanted to determine if the CaM constructs were expressed at similar levels in the cells. Immunoblot analysis indicated that there was variation in the level of expression of the constructs, however the expression levels of the CaM-constructs do not appear to play a significant role in their effects, since CaM-C-term and CaM-center had similar beneficial effects but immunoblot analysis indicated disparate expression levels (FIG. 5A). Furthermore, CaM-N-term had intermediate levels of expression on immnoblot but had dissimilar effects compared to CaM-center and CaM-C-term. However, due to the small size of the CaM-constructs detection of the proteins on immunoblots was difficult. In fact we were unable to efficiently detect CaM-overlap. This limitation may account for the differences in expression that were observed on the immunoblots. Therefore, we also examined the expression levels of the constructs via immunocytochemistry. The percentage of immunopositive cells and staining intensity were similar for all the constructs suggesting that they are expressed at similar levels. Huntingtin with an expanded glutamine repeat has the propensity to aggregate in the cytoplasm as well as the nucleus of cells. Therefore we wanted to determine if these small CaM constructs are expressed in both cellular locations. HEK-293T cells were transfected with the CaM constructs and immunocytochemistry was performed. All four CaM constructs, CaM-overlap, CaM-N-term, CaM-C-term, CaM-center, were found to be expressed in both the cytoplasm as well as the nucleus of cells (FIG. 5B). This suggests that the CaM constructs can have an effect both in the cytoplasm as well as the nucleus.

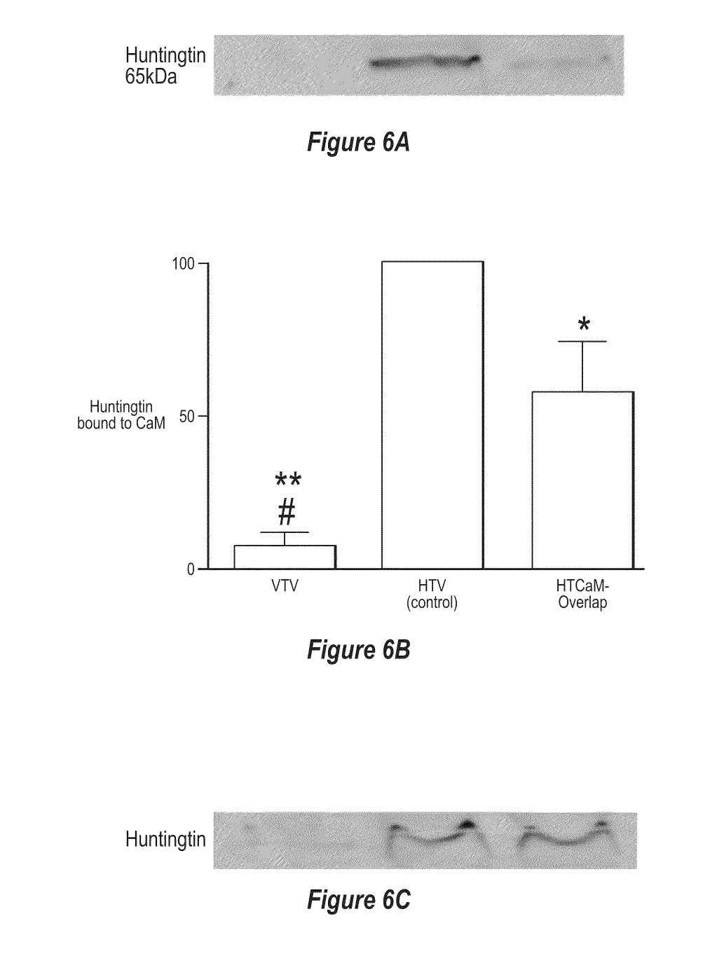

It was found that CaM-overlap inhibits calmodulin-huntingtin interaction. The next experiments were designed to further investigate CaM-overlap and determine the possible mechanism for the effects of expression of CaM-overlap on TG-modified huntingtin, cytotoxicity and Ca²⁺ release. Since it was previously shown that huntingtin from cell lysates expressing htt-N63-148Q and TG2 bound to CaM, we wanted to determine if the CaM-overlap construct could inhibit this huntingtin-CaM interaction. HEK-293T cells were transfected with htt-N63-148Q, TG2 and either vector or CaM-overlap and 48 hours post transfection cells were harvested. Cell lysates were incubated with CaM-agarose and bound proteins were examined on immunoblots. Huntingtin proteins were detected on the immunoblots in order to determine the amount of huntingtin that bound to CaM. Cells expressing CaM-overlap had less huntingtin bound to CaM as compared to control cells expressing vector (FIGS. 6A-6C), indicating that the CaM-overlap peptide can interrupt the interaction between mutant huntingtin and CaM.

In sum, these data support the hypothesis that the interaction of TG, CaM and mutant huntingtin may impede the normal activity of TG and CaM proteins resulting in neurotoxic effects in HD, and inhibiting the interaction of CaM with mutant huntingtin protein is a potential target for therapeutic intervention in HD.

### Example Set B Protocols

Adeno-associated virus vector construction was performed as described herein. A fragment of exons 4 and 5 of the calmodulin gene, encoding amino acids 76-121 of calmodulin (KDTDSEEEIREAFRVFDKDGNGYISAAELRHVMTNLGEKLTDEEV (SEQ ID NO: 1) (CaM-peptide)), was cloned into pEF6. The CaM-peptide was randomly scrambled to prepare a control amino acid sequence: GDTVEREKDAYNSLEGFDNTIHTLRADIGMVEEVAKSKRDEEFLE (SEQ ID NO: 23) (scram-CaM-peptide). The scrambled sequence was then analyzed at the SIB site using the BLAST network service to identify sequence similarity with the sequences of other proteins. Once it was determined that there was no significant similarity with any other protein sequence, the sequence was synthesized and cloned into pZERO-2 vector (IDT DNA Technologies, Coralville, Iowa). The pEF6 vector encoding CaM-peptide and the pZERO-2 vector encoding scram-CaM-peptide were used as templates in a multi-step PCR method. CaM-peptide or scram-CaM-peptide was amplified using primers 1A and 2A or 4A and 5A, respectively, containing XhoI and EcoRI restriction sites. The resultant PCR products were digested and cloned into the XhoI and EcoRI sites in the pMig vector (generous gift from Dr. Vinay Kumar, University of Chicago), which encodes for an internal ribosome entry site (IRES) followed by green fluorescent protein (GFP). The resultant CaM-peptide-IRES-GFP and scram-CaM-peptide-IRES-GFP pMig vectors were used as templates to amplify CaM-peptide-IRES-GFP and scram-CaM-peptide-IRES-GFP using primers 1A and 3A, and 4A and 3A, respectively, containing XhoI and KpnI restriction sites. The resultant PCR products were digested and cloned into the XhoI and KpnI sites in the pGAN vector (Gene Transfer Vector Core, University of Iowa, Iowa City, Iowa) containing CMV promoter and BGH polyA. The resultant CMV-CaM-peptide-IRES-GFP and CMV-scram-CaM-peptide-IRES-GFP pGAN vectors were then subcloned into NotI restriction sites in the pFBGR vector (Gene Transfer Vector Core, University of Iowa, Iowa City, Iowa) containing adeno-associated viral elements. Constructs were verified by restriction digests and sequencing. The resultant vectors as well as an empty pFBGR vector, which encodes for GFP only, were then used for production of the adeno-associated virus 2 (AAV) expressing either CaM-peptide and GFP (AAV-CaM-peptide+GFP), scram-CaM-peptide and GFP (AAV-scram-CaM-peptide+GFP), or only GFP (AAV-GFP) (Gene Transfer Vector Core, University of Iowa, Iowa City, Iowa).

PCR primers used for generating vectors:

(SEQ ID NO: 24)

| | |
|---|---|
| Primer 1A: | ACTCGACTCGAGATCATGAAGGACACAGACAGTGAG |

(SEQ ID NO: 25)

| | |
|---|---|
| Primer 2A: | ACTCGAGAATTCCTACACCTCCTCATCGGTCAGCTTC |

(SEQ ID NO: 26)

| | |
|---|---|
| Primer 3A: | ACTCGAGGTACCTTACTTGTACAGCTCGTCCATGCC |

(SEQ ID NO: 27)

| | |
|---|---|
| Primer 4A: | ACTCGACTCGAGATCATGGGCGATACCGTGGAACG |

(SEQ ID NO: 28)

| | |
|---|---|
| Primer 5A: | ACTCGAGAATTCCTATTCCAGAAATTCTTCATCACG |

Cell culture. Human SH-SY5Y cells were grown at 37° C. in RPMI 1640 medium (Invitrogen, Carlsbad, Calif.) containing 4 mM glutamine, 5% FBS, 10% horse serum, 100 units/ml penicillin, and 100 μg/ml streptomycin in the presence of 5% CO₂. Cells were grown to 80-90% confluency and transfected with a 63 amino acid N-terminal huntingtin fragment containing 148 polyglutamine repeats (htt-N63-148Q) using Lipofectamine Plus (Invitrogen, Carlsbald, Calif.). Cells stably expressing htt-N63-148Q were selected based on their resistance to blasticidin. Cells were treated with 10 μM retinoic acid (Sigma, St. Louis, Mo.) for 4 days to induce differentiation and upregulate transglutaminase expression (Tucholski J, Lesort M, Johnson G V (2001) Tissue transglutaminase is essential for neurite outgrowth in human neuroblastoma SH-SY5Y cells. Neuroscience.102(2):481-91) and then were infected with either AAV-CaM-peptide+GFP, AAV-scram-CaM-peptide+GFP or AAV-GFP at a multiplicity of infection (MOI) of 50.

Fluorescence microscopy. Cells were infected with either AAV-CaM-peptide+GFP, AAV-scram-CaM-peptide+GFP or AAV-GFP and plated onto collagen-coated chamber slides. Forty-eight hours post-infection, cells were washed in PBS and fixed in 4% paraformaldehyde for 20 minutes at room temperature, then washed again in PBS. Coverslips were mounted with media containing DAPI. Cells were examined as described herein.

Flow Cytometry. Forty-eight hours post viral infection, cells were harvested and centrifuged at 1500 g for 5 min. Cell pellets were washed twice in PBS with 1% BSA. The cells pellets were then resuspended in 300 μl PBS with 1% BSA and were analyzed on a GFP channel using BD FACS caliber flow cytomer (BD Biosciences). Data were analyzed on the summit software suite (Dako Cytomation, Carpenteria, Calif.).

Immunoprecipitation. Forty-eight hours post viral infection, cells were harvested and resuspended in lysis buffer containing 50 mM Tris-HCl, pH 8.8, 100 mM NaCl, 5 mM MgCl₂, 0.05% NP-40, 1 mM EDTA (Hazeki N, Tukamoto T, Goto J, Kanazawa I (2000) Formic acid dissolves aggregates of an N-terminal huntingtin fragment containing an expanded polyglutamine tract: applying to quantification of protein components of the aggregates. Biochem Biophys Res Commun. 277(2):386-93), and 1:1,000 protease inhibitor mixture (Sigma, St. Louis, Mo.). Insoluble fractions were prepared by centrifuging the cell lysates at 12,000×g for 5 minutes, removing the supernatant, resuspending the pellet in 95% formic acid, and incubating at 37° C. for 40 minutes. The formic acid was then removed under vacuum. The pellets were then resuspended in 10 mM Tris-HCl, pH 7.5, 0.14 M NaCl, and 0.1% Tween 20, for further analysis. Immunopurification of proteins containing transglutaminase-catalyzed ε-(γ-glutamyl)lysine bonds was performed as described herein. The eluted immunopurified proteins were stored at −80° C. until immunoblot analysis.

Antibodies. The mouse anti-myc monoclonal described above was used as described herein. The mouse anti-actin antibody was used at 1:10,000 for immunoblots (MP Biomedicals, Solon, Ohio) in order to detect actin.

Ex vivo transglutaminase assay. In this ex vivo assay, transglutaminase activity is measured in cell lysates. 96-well Immulon 4 HBX plate (Dynatech, Franklin, Mass.) was coated with N—N dimethylcasein (Sigma, St. Louis, Mo.) in sodium bicarbonate overnight at 4° C. Then the plates were washed with PBS and blocked with 2% milk in PBS for 1 hour at 37° C., followed by 3 washes with PBS. Forty-eight hours post viral infection, cells were harvested and lysed in 0.1 M Tris-HCl pH 8.3, 1 mM EDTA, 1 mM PMSF, and protease inhibitor cocktail. Cell lysates (25 μg of protein/sample) were diluted in 0.1 M Tris-HCl pH 8.5, 0.15 M NaCl, 5 mM DTT, 0.5 mM biotin labeled amine, and 5 mM CaCl₂. The mixture was then added to the wells in triplicate and incubated for 1 hour at 37° C. The plate was washed with TBS and 0.001% Tween followed by incubation with streptavidin conjugated to HRP (Jackson ImmunoResearch, West Grove, Pa.) for 1 hour at room temperature. After a final wash, 1×TMB substrate solution (eBioscience, SanDiego, Calif.) was added and color was allowed to develop for 5-10 minutes. Then the reaction was stopped with 3 N sulfuric acid and the absorbance was read at 450 nm.

In situ transglutaminase assay. In this in situ assay, transglutaminase activity is measured in intact cells via incorporation of a polyamine and the presence of the transglutaminase-catalyzed bond. Forty-two hours post viral infection, cells were treated with 2 mM 5-(biotinamido)pentylamine, a biotinylated polyamine (Pierce, Rockford, Ill.), and 6 hours post polyamine treatment, cells were harvested and lysed in 0.1 M Tris-HCl pH 8.3, 1 mM EDTA, 1 mM PMSF, and protease inhibitor cocktail. 96-well Immulon 4 HBX plates (Dynatech, Franklin, Mass.) were coated with anti-ε-(γ-glutamyl)lysine (81D4) antibody (CovalAb, Lyon, France) in 0.1 M Na₂HPO₄.7 H₂O pH 9 overnight at 4° C. Then the plates were washed with PBS and blocked with 2% milk in PBS for 1 hour at room temperature, followed by 3 washes with PBS with 0.0375% Tween. Cell lysates (50 μg of protein/sample/well) were added to the wells in triplicates and incubated for 1 hour at 37° C. The plate was washed with PBS and 0.0375% Tween, followed by incubation with streptavidin conjugated to HRP (Jackson ImmunoResearch, West Grove, Pa.) for 1 hour at room temperature. After final washes, a 1×TMB substrate solution (eBioscience, SanDiego, Calif.) was added and color was allowed to develop for 5-10 minutes. The reaction was stopped with 3 N sulfuric acid and the absorbance was read at 450 nm.

CaM Kinase II Activity Assay. CaM kinase II enzyme activity was analyzed using the SignaTECT Calcium/Calmodulin-Dependent Protein Kinase Assay System (Promega, Madison, Wis.), according to the manufacturer's protocol as well as without the addition of exogenous CaM. Briefly, SH-SY5Y cells were transfected with vector (V), htt-N63-148Q (H)+V, CaM-peptide+V, or H+CaM-peptide using Lipofectamine Plus (Invitrogen, Carlsbald, Calif.). Forty-eight hours post transfection cells were lysed in CaM kinase extraction buffer (20 mM Tris-HCl, pH 8.0, 2 mM EDTA, 2 mM EGTA, 2 mM DTT, 1 mM PMSF and 1:1000 Protease Inhibitor Cocktail (Sigma, St Louis, Mo.)). Cell lysates were mixed with [γ-³²P]ATP (at 3000 Ci/mmol, 10 mCi/ml), activation buffer containing 5 mM CaCl₂ and 5 μM calmodulin, or control buffer containing 5 mM EGTA, with and without biotinylated CaM kinase II substrate. After incubation at 30° C. for 2 minutes, the reaction was terminated by adding 7.5 M guanidine hydrochloride and spotted to a streptavidin-impregnated membrane. Membranes were washed and retained radioactivity was quantified by liquid scintillation counting (Beckman, Fullerton, Calif.). Radioactive counts were converted to endogenous CaM kinase II activity in the sample by the following formula:

$$Enzyme activity ( in p mol \text{/} min \text{/} µg of protein ) = \frac{( \begin{matrix}{cpm}_{reaction with calcium / calmodulin} - \\ {cpm}_{reaction without calcium / calmodulin}\end{matrix} ) \times ( 37.5 )}{\begin{matrix}20 \times ( amount of protein in {reaction}_{μ g} ) \times \\ ( specific activity of [ γ {-}^{32} P ] ATP )\end{matrix}}$$

Expression and purification of recombinant mutant huntingtin protein. Escherichia coli expressing a modified pMAL vector (New England Biolabs, Ipswich, Mass.) encoding exon 1 of huntingtin with 44 glutamine residues (kindly provided by Dr. Christopher Ross, The Johns Hopkins University School of Medicine, Baltimore, Md.) were grown to an OD₆₀₀ of 0.6-0.8, induced by adding 0.3 mM isopropyl-β-D-thiogalactopyranoside followed by shaking for 2 hours at 28° C., and then centrifuged for 15 min at 5,000 g. The cell pellets were then resuspended in lysis buffer (PBS pH 7.4, supplemented with 10 mM methionine, 2 mM EDTA, 5 mM dithiothreitol, 1 mM 4-(2-aminoethyl)benzenesulfonyl fluoride, and protease inhibitor mixture (Sigma, St. Louis, Mo.)), and samples were frozen in a dry ice-ethanol mixture. Samples were then thawed on ice followed by sonication and then centrifuged at 20,000×g for 15 min. To purify MBP fusion proteins, the supernatant was applied to an amylose resin (New England Biolabs, Ipswich, Mass.) packed column (2.5×10 cm) that had first been washed with 8 column volumes of PBS pH 7.4. After addition of the lysate supernatant, the column was then washed with 12 column volumes of PBS pH 7.4, followed by elution of the fusion protein with 2 column volumes of PBS with 10 mM maltose. The fusion protein was further purified by incubating with Ni—NTA agarose (Qiagen, Valencia, Calif.) in PBS supplemented with 10 mM imidazole with rocking for 2 hours at 4° C. The resin was then washed 3 times with PBS supplemented with 20 mM imidazole, and fusion protein was eluted with 6 resin volumes of PBS supplemented with 250 mM imidazole. Purified fusion protein was analyzed by SDS-PAGE and visualized by Coomassie staining.

In vitro mutant huntingtin-calmodulin binding. Binding of purified mutant huntingtin to calmodulin was studied using calmodulin-agarose (Sigma, St. Louis, Mo.). Purified mutant huntingtin protein (8.4 μg/ml) or purified calcineurin protein (1.4 μg/mL, positive control) (Sigma, St. Louis, Mo.), with or without varying concentrations of purified CaM-overlap peptide (Biopeptide Co. Inc., San Diego, Calif.) and/or varying concentrations of N-(6-Aminohexyl)-1-naphthalenesulfonamide hydrochloride (W-5), a calmodulin antagonist (Tocris, Ellisville, Mo.)), were mixed with 45 μg/ml calmodulin-agarose in 10 mM Tris-HCl (pH 8) containing 1 mM CaCl₂ and 150 mM NaCl, and then incubated overnight at 4° C. After washing three times with 20 mM Hepes pH 7.4, 150 mM NaCl, 5 mM sodium pyrophosphate, 10% glycerol, 1% Triton X-100, 1 mM CaCl₂, and protease inhibitor mixture (Sigma, St. Louis, Mo.), and once with 10 mM Hepes pH 7.4, the agarose-bound proteins were eluted by incubation at 100° C. for 10 minutes in Laemmli sample loading buffer. The fractions obtained were analyzed by SDS-gel electrophoresis followed by immunoblotting.

### Example Set B Results

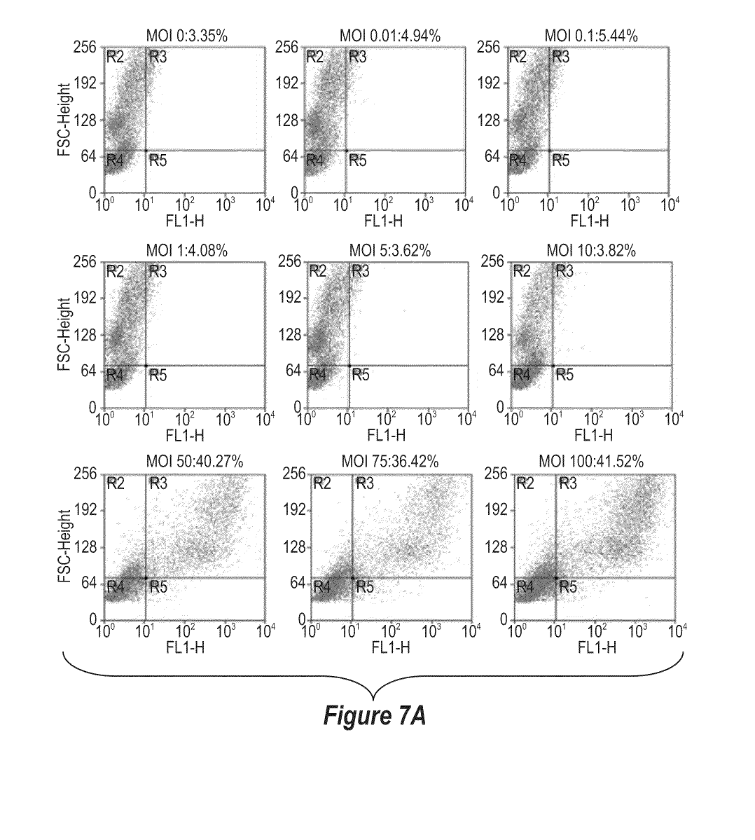

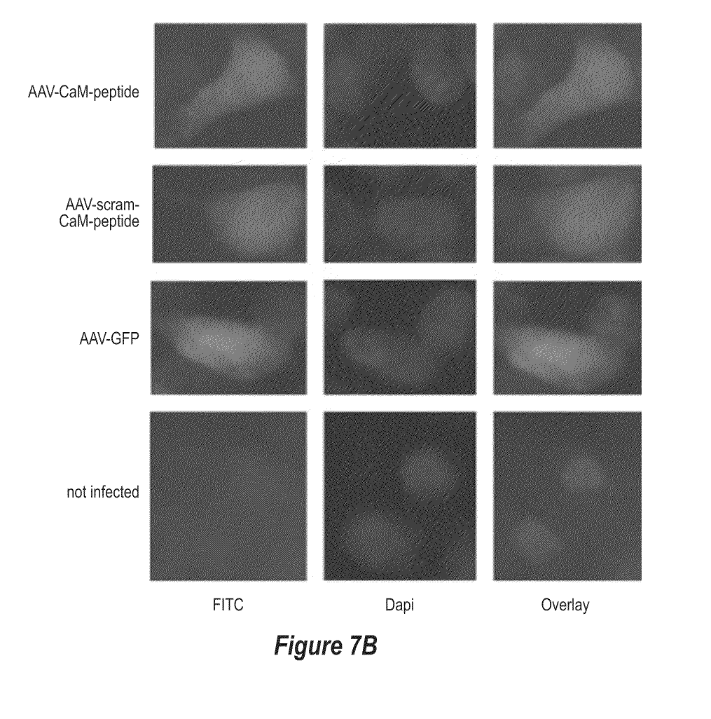

It was found that AAV-mediated expression of CaM-peptide in human neuroblastoma SH-SY5Y cells can stably express N-terminus of mutant huntingtin. We developed three different adeno-associated viruses (AAV): (1) an AAV which mediates expression of a peptide consisting of amino acids 76-121 of CaM (CaM-peptide) along with GFP (AAV-CaM-peptide+GFP); (2) an AAV which mediates expression of a scrambled version of CaM-peptide along with GFP (AAV-scram-CaM-peptide+GFP); and (3) an AAV which mediates expression of only GFP (AAV-GFP). In order to determine an appropriate multiplicity of infection (MOI) for the AAV, SH-SY5Y cells were transduced with varying MOIs (e.g., 0, 0.01, 0.1, 1, 5, 10, 50, 75, 100) of AAV-GFP. Forty-eight hours post-infection cells were assessed for AAV-mediated GFP expression using flow cytometry (FIG. 7A). A MOI of 50 resulted in the largest percentage of GFP positive SH-SY5Y cells with the lowest level of cell death. To determine if a MOI of 50 resulted in similar intracellular distribution and expression of GFP in differentiated SH-SY5Y cells for all three AAV developed, we performed fluorescence microscopy. A MOI of 50 for each of the viruses showed similar AAV-mediated GFP expression in differentiated SH-SY5Y cells (FIG. 7B).

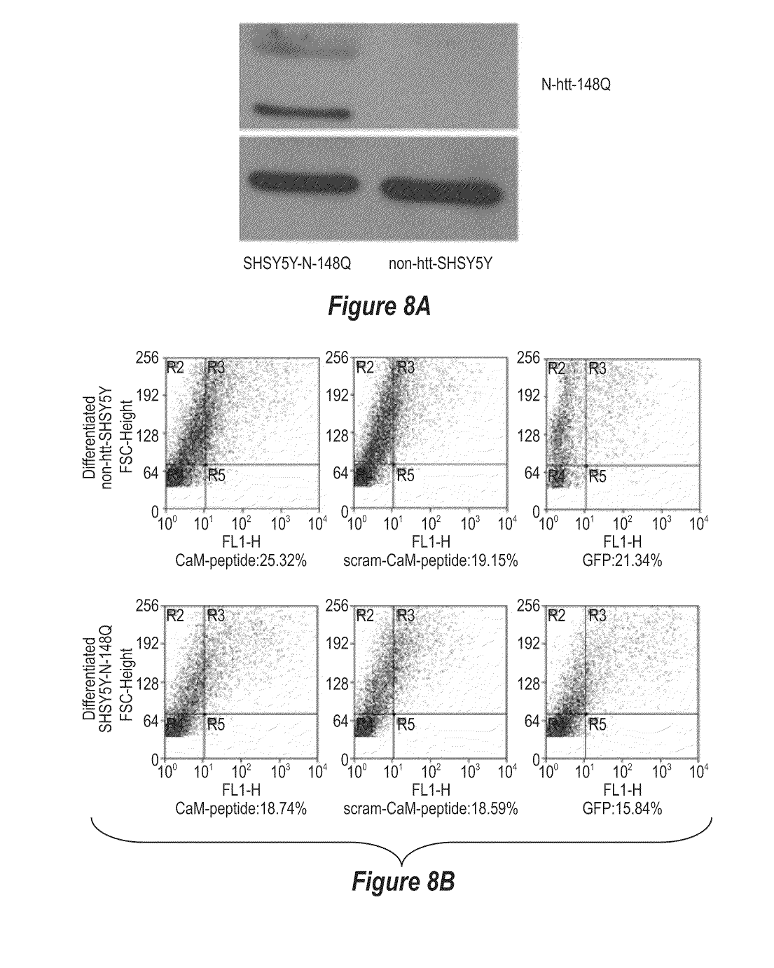

Next, we developed human neuroblastoma SH-SY5Y cell lines that stably express N-terminal mutant huntingtin. Expression levels of each cell line were estimated by Western blot analysis, and cell lines that showed moderate and similar expression were selected (FIG. 8A). To further investigate the viral infection procedure, we used flow cytometry to quantitatively determine if a MOI of 50 would result in similar levels of infection for all 3 AAV (AAV-CaM-peptide+GFP, AAV-scram-CaM-peptide+GFP or AAV-GFP) in differentiated SH-SY5Y cells not expressing mutant huntingtin (non-htt-SHSY5Y cells) and in differentiated SH-SY5Y cells that stably express mutant huntingtin (SHSY5Y-htt-N63- 148Q cells). Non-htt-SHSY5Y and SHSY5Y-htt-N63-148Q cells were treated with 10 μM retinoic acid for 4 days to induce differentiation. Then cells were infected with either AAV-CaM-peptide+GFP, AAV-scram-CaM-peptide+GFP, or AAV-GFP (MOI=50). Forty-eight hours post-infection, cells were assessed for GFP expression by flow cytometry. All three viruses infected differentiated non-htt-SHSY5Y and SHSY5Y-htt-N63-148Q cells with similar efficiencies (FIG. 8B). However, these values were lower than previously measured for this MOI, perhaps due to conditions associated with neuronal differentiation.

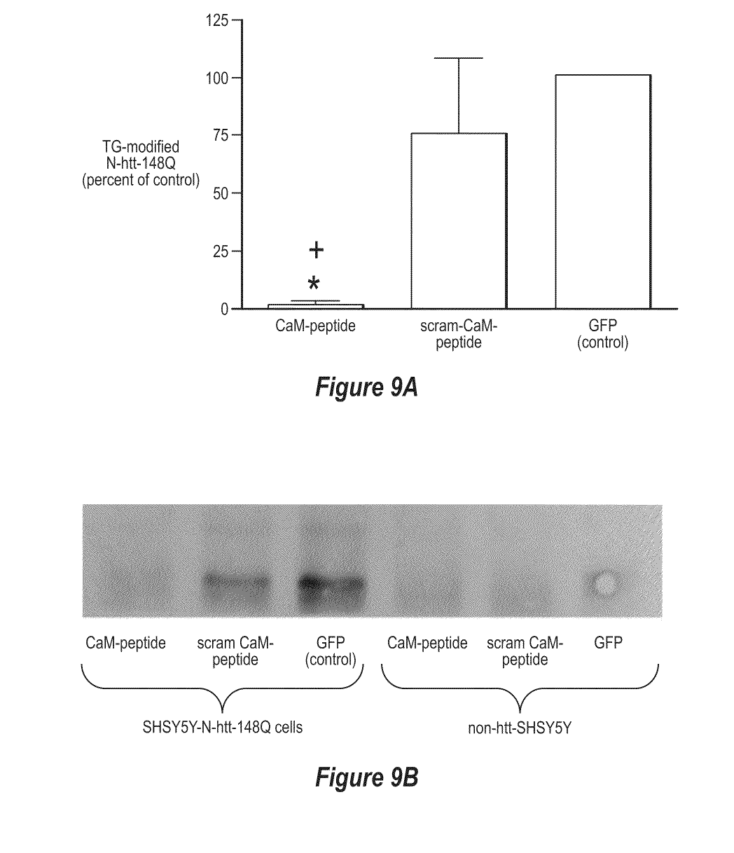

It was found that AAV-mediated expression of CaM-peptide decreases TG-catalyzed modifications of mutant huntingtin in SH-SY5Y-htt-N63-148Q cells. With the cell and viral system now defined, we determined the effect of viral-mediated expression of CaM-peptide in differentiated SHSY5Y-htt-N63-148Q cells on TG-catalyzed modifications of N-terminal mutant huntingtin. Cells were differentiated, infected with either AAV-CaM-peptide+GFP, AAV-scram-CaM-peptide+GFP or AAV-GFP (MOI=50), and forty-eight hours post-infection cells were assayed for TG-modified N-terminal mutant huntingtin. SHSY5Y-htt-N63-148Q cells expressing CaM-peptide had a significantly lower amount of TG-modified N-terminal huntingtin compared to cells expressing scram-CaM-peptide or only GFP (FIGS. 9A-9B).

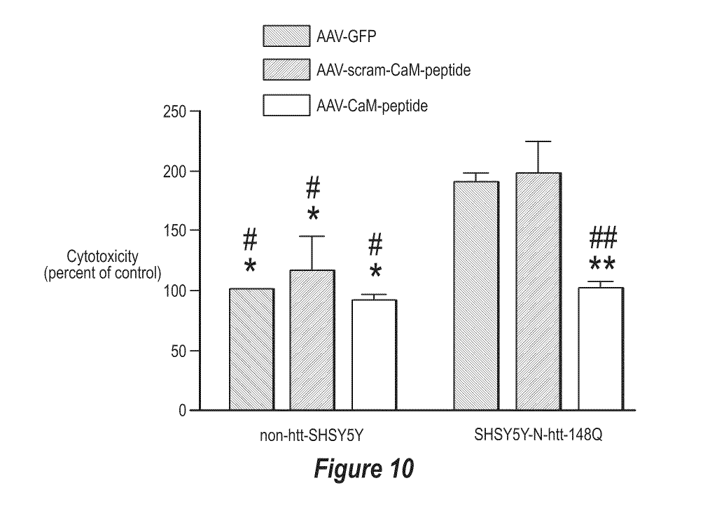

It has found that AAV-mediated expression of CaM-peptide decreases mutant huntingtin associated cytotoxicity in neuroblastoma cell lines that express N-terminal mutant huntingtin. Differentiated non-htt-SHSY5Y or SHSY5Y-htt-N63-148Q cells were infected with either AAV-CaM-peptide+GFP, AAV-scram-CaM-peptide+GFP, or AAV-GFP (MOI=50), and forty-eight hours post-infection levels of cytotoxicity were measured. SHSY5Y-htt-N63-148Q cells expressing CaM-peptide had approximately half the level of cytotoxicity compared to SHSY5Y-htt-N63-148Q cells expressing scram-CaM-peptide or only GFP. Furthermore, there was no significant difference in the level of cytotoxicity between SHSY5Y-htt-N63-148Q cells expressing CaM-peptide and non-htt-SHSY5Y cells (FIG. 10).

Differentiated SHSY5Y cells transiently transfected with either htt-N63-18Q, htt-N63-148Q were infected with either AAV-CaM-peptide+GFP, or AAV-scram-CaM-peptide+GFP, or were uninfected (MOI=50), and ninety six hours post-infection levels of cytotoxicity were measured using the LDH assay. In cells transiently transfected with htt-N63-18Q, we found that infection with AAV-CaM-peptide had no effect on cytotoxicity (38.1±1.1; mean percent cytotoxicity±S.E.M.) compared to cells infected with AAV-scram-CaM-peptide (35.4±2.4) or uninfected cells (38.0±1.3). However, in these transiently transfected cells, there was also no increase in cytotoxicity in cells transfected with htt-N63-148Q (34.6±2.3) compared to cells transfected with htt-N63-18Q (38.0±1.3). The lack of toxicity of the htt-N63-148Q was perhaps due to the lower levels of expression of the htt-N63 constructs in transiently transfected cells compared to stable transfections.

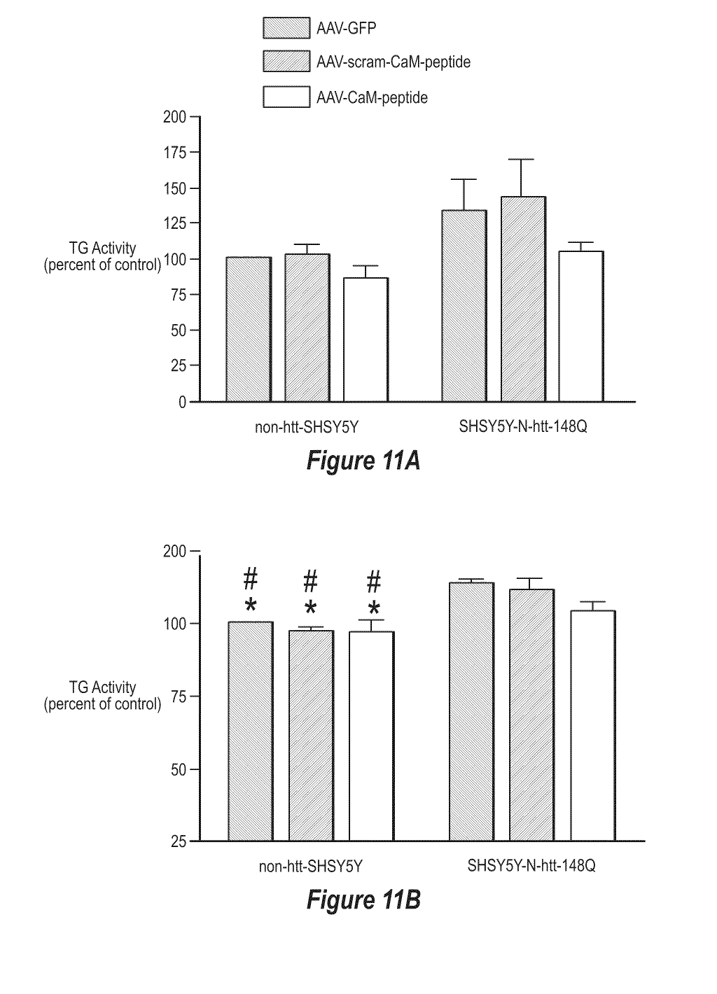

The effect of AAV-mediated expression of CaM-peptide on total transglutaminase activity in neuroblastoma cell lines that stably express N-terminal mutant huntingtin was studied. We next examined the effect of expression of the CaM-peptide on total TG-activity in differentiated non-htt-SHSY5Y and SHSY5Y-htt-N63-148Q cells. Cells were infected with AAV-CaM-peptide+GFP, AAV-scram-CaM-peptide+GFP, or AAV-GFP (MOI=50), and harvested forty-eight hours post-infection. TG activity was measured ex vivo based on the incorporation of 5-(biotinamido)pentylamine into the N,N′-dimethylcasein substrate coated onto micro-plates. There were no significant differences in total TG activity between the different infections (AAV-CaM-peptide+GFP, AAV-scram-CaM-peptide+GFP or AAV-GFP (control)) in either non-htt-SHSY5Y cells or SHSY5Y-htt-N63-148Q cells (FIG. 11A). There were small but insignificant increases in total TG activity in SHSY5Y-htt-N63-148Q cells expressing scram-CaM-peptide or only GFP, compared to non-htt-SHSY5Y cells expressing either GFP only, scram-CaM-peptide, or CaM-peptide (FIG. 11A). To determine whether cell lysis had an effect on enzyme activity, we measured total TG activity in situ. Non-htt- SHSY5Y and SHSY5Y-htt-N63-148Q cells were infected with one of the various AAV, and forty-two hours post-infection, were treated with 5-(biotinamido)pentylamine. Six hours later cells were harvested and the cell lysates were applied to micro-plates coated with anti-ε-(γ-glutamyl)lysine (81D4) antibody in order to capture proteins that were modified by TG in situ. TG-catalyzed incorporation of 5-(biotinamido)pentylamine into cellular proteins was then determined by incubation with streptavidin-HRP. Similar to the ex vivo assay, there was no significant difference in total TG activity between the various infections (CaM-peptide, scram-CaM-peptide, or GFP (control)) in SHSY5Y-htt-N63-148Q cells (FIG. 11B). Similarly, there also was no difference in total TG activity in the various infections in non-htt-SHSY5Y cells. However, there was a significant decrease in total TG activity in non-htt-SHSY5Y cells compared to SHSY5Y-htt-N63-148Q cells expressing scram-CaM-peptide or only GFP. Total TG activity in non-htt-SHSY5Y cells was not significantly different from total TG activity in SHSY5Y-htt-N63-148Q cells expressing CaM-peptide (FIG. 11B).

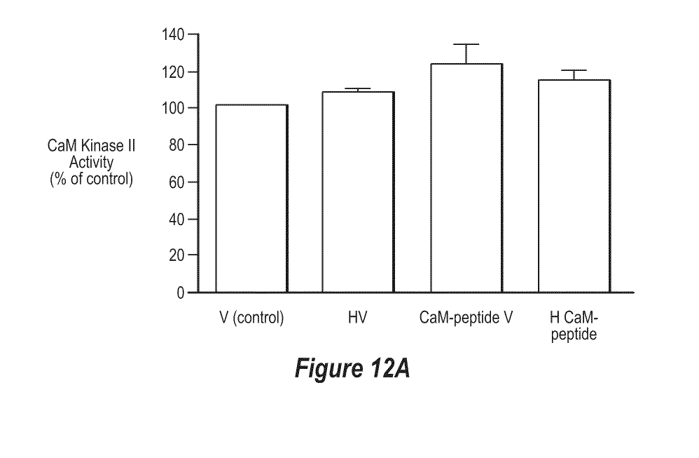

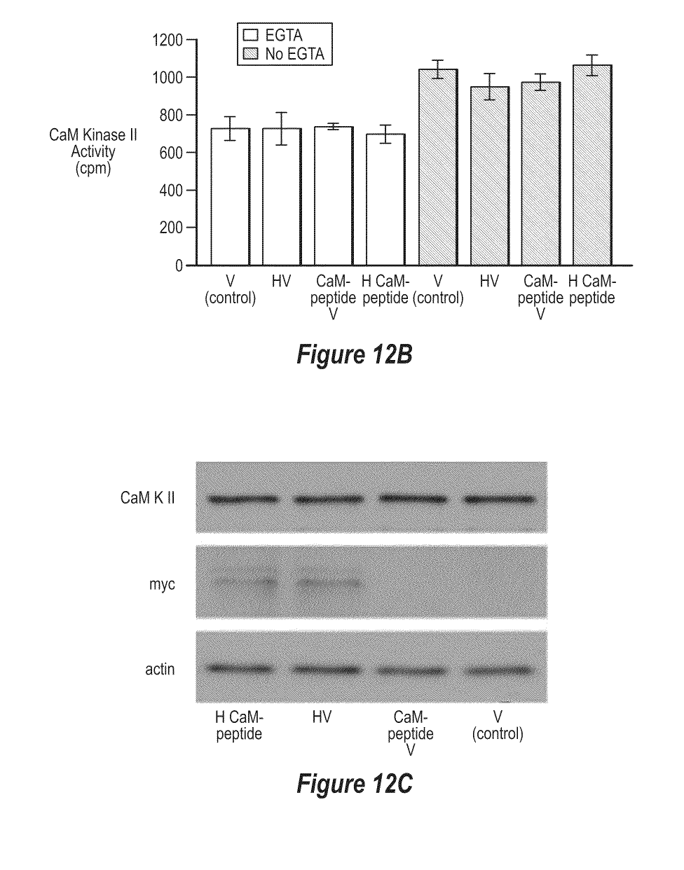

It has been found that CaM-peptide does not significantly affect CaM kinase II activity. To determine if expression of CaM-peptide has effects on the activity of other CaM-dependent enzymes, we examined whether expression of CaM-peptide affects calmodulin-dependent protein kinase II (CaM kinase II) activity. SH-SY5Y cells were transfected with vector, htt-N63-148Q, CaM-peptide or the combination of htt-N63-148Q and CaM-peptide. We found no significant differences in CaM kinase II activity among the various transfections (FIGS. 12A-12C). Interestingly, expression of CaM-peptide alone resulted in a small but insignificant increase in CaM kinase II activity compared to all other transfections. We also examined the activity of CaM kinase II without the addition of exogenous CaM (addition of exogenous CaM is recommended as described in the manufacturer's protocol). There was no significant effect of CaM-peptide under these assay conditions either (FIGS. 12A-12C).

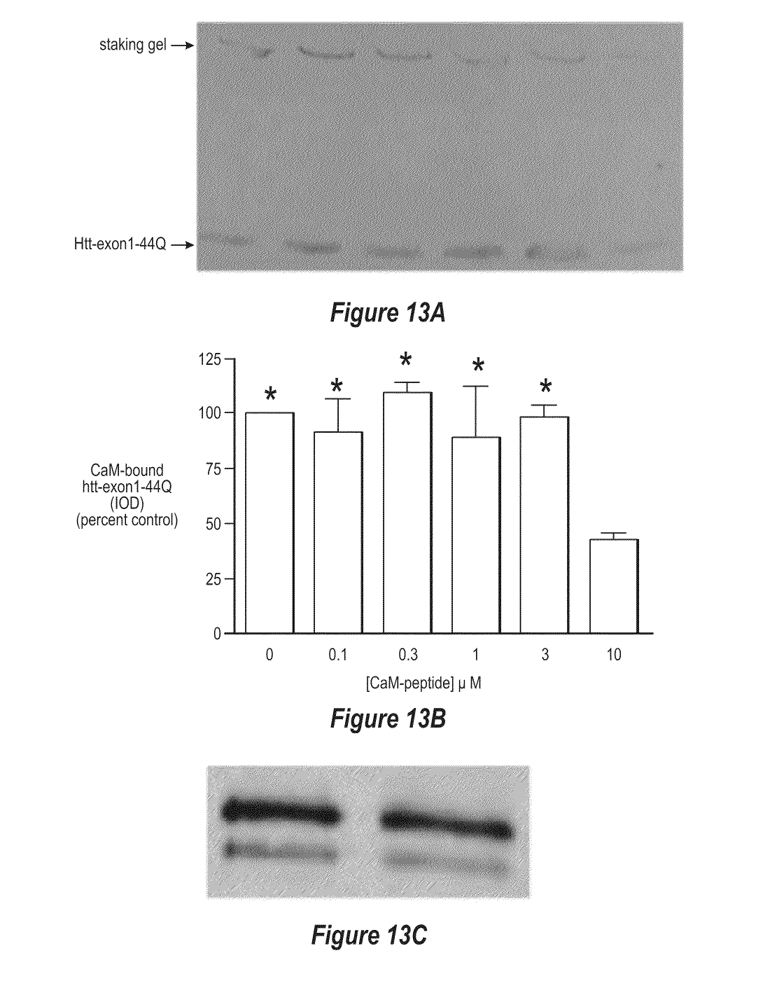

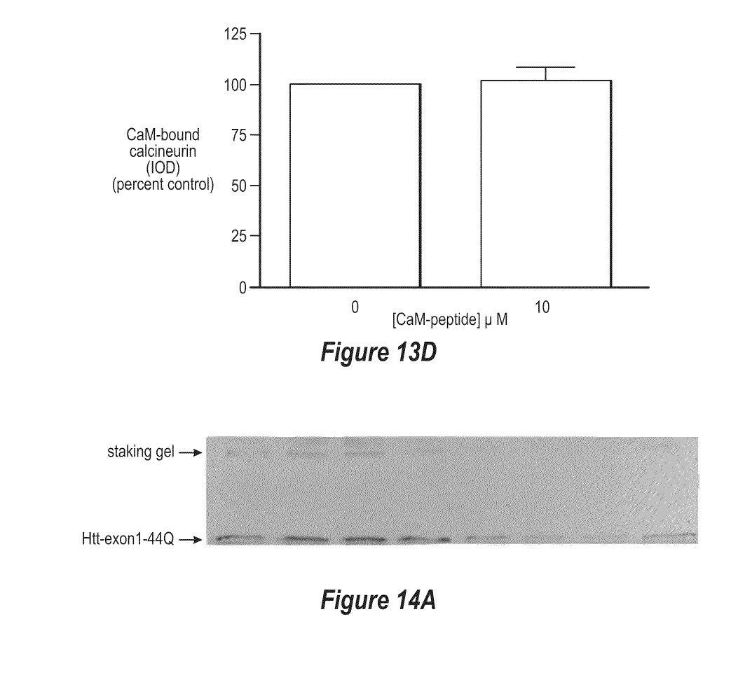

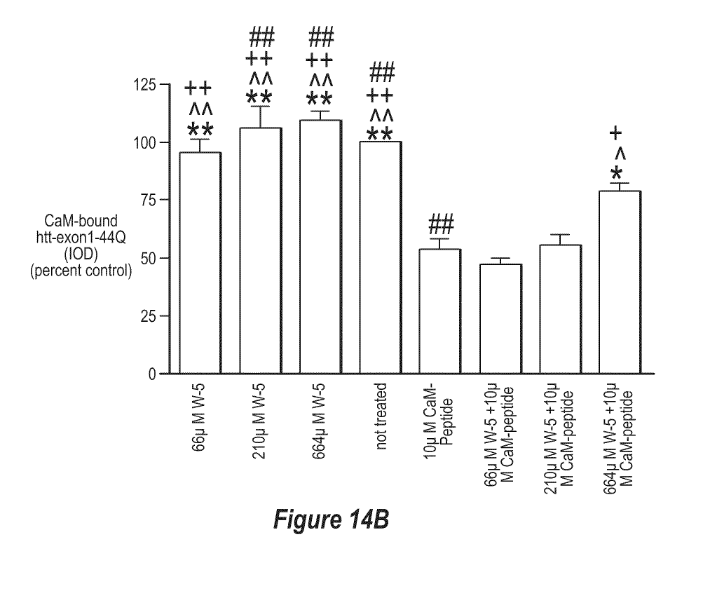

It has been found that CaM-peptide inhibits binding of N-terminal mutant huntingtin with an expanded polyglutamine repeat and CaM. Thus far all the experiments performed to examine the effects of CaM-peptide were done in cells where other endogenous proteins could play a role in mediating the action of CaM-peptide. Therefore an in vitro assay was used to determine if CaM-peptide could directly interfere with the interaction of N-terminal mutant huntingtin and CaM. CaM-agarose was used to immunoprecipitate recombinant purified huntingtin exon 1 with an expanded polyglutamine repeat (htt-exon1-44Q) in the absence and presence of varying concentrations of CaM-peptide. The amount of CaM-bound htt-exon1-44Q was significantly lower when 10 μM of CaM-peptide was present than when CaM-peptide was absent (FIGS. 13A and 13B). To determine if CaM-peptide would non-specifically inhibit the interaction of CaM with any CaM-binding protein, we incubated calcineurin with CaM-agarose in the absence and presence of 10 μM of CaM-peptide. The presence of 10 μM of CaM-peptide did not affect the binding of CaM with calcineurin (FIGS. 13C and 13D). Next, to investigate the potential site of interaction of CaM-peptide the CaM antagonist, W-5, was used along with CaM-peptide. W-5 alone did not affect the amount of CaM-bound htt-exonl-44Q, but once again the amount of CaM-bound htt-exonl-44Q was significantly lower when 10 μM of CaM-peptide was present. However, the amount of CaM-bound htt-exon1-44Q was significantly increased when 664 μM W-5 is present along with 10 μM of CaM-peptide (FIGS. 14A-14B).

### Example Set C Protocols

Animals. Male R6/2 transgenic mice (with 100-115 CAG repeats in the transgene) and wild-type littermate mice were purchased from Jackson Laboratories (Bar Harbor, Me.) at 6 weeks of age. A reduction in the number of CAG repeats from the original approximately 154-159 to 100-115 was found in 2006, along with a delay in the age of onset and a decrease in the severity of the neurological phenotype. The delayed onset of the neurological phenotype suggests that treatment at 7 weeks of age should be sufficient to determine the treatment effect. The mice had free access to food and water in an environment controlled for temperature and humidity and a 12 h light/dark cycle. The behavioral tests were conducted in the light part of the cycle. All procedures were conducted in accordance with the National Institutes of Health Guide for the Care and Use of Laboratory Animals as approved by the University of Kansas Institutional Animal Care and Use Committee.

Recombinant AAV serotype 2 vectors were generated as described above. Three resultant vectors expressed each of the following: GFP and the CaM-fragment (containing amino acids 76-121 of calmodulin), GFP and a scrambled peptide (containing the same amino acids of the CaM-fragment but in a randomly scrambled sequence), and GFP alone. The titers of final AAV products ranged from 2˜5×10¹⁴ vector genomes per ml. Bilateral striatal injections of GFP-expressing AAV were performed in 7-week-old mice (1 μl per each side with 200 nl/min infusion rate at coordinates of anterior/posterior 0.5 mm, lateral/medial ±2 mm and dorsal/ventral −3 mm relative to bregma (Franklin et al., 1997)). Mice were randomly divided into five groups (11˜14 mice for each group) with a defined genotype and treatment: CaM-HD (R6/2 mice injected with AAV expressing the CaM-fragment), Vec-HD (R6/2 mice injected with empty vector AAV expressing GFP alone), Scr-HD (R6/2 mice injected with AAV expressing a scrambled version of the CaM-fragment), CaM-WT (wild-type mice injected with AAV expressing CaM-fragment) and Vec-WT (wild-type mice injected with empty vector AAV).

Body weight and survival. Body weights were measured twice a week. Survival was checked twice daily at 8 h intervals. Brains were removed after death and snap frozen in 2-methylbutane/dry ice bath and stored at −80° C.

Behavioral tests. Three behavioral tests were performed between 10 and 14 weeks of age at weekly intervals. Rotarod performance, gait dynamics and locomotion were examined every Wednesday, Friday and Sunday, respectively.

Gait dynamics. The DigiGait imaging system (Mouse Specifics, Inc., Boston, Mass.) was utilized for gait analyses as previously described (Hampton et al., 2004). Briefly, mice were placed on a motorized transparent treadmill belt moving at a speed of 14 cm/s. Digital images of paw placement were recorded at 150 Hz through a video camera mounted below the animal. The proprietary software analyzed the resulting digital images to generate a set of periodic waveforms that described the paw area and movement of each limb relative to the treadmill belt through consecutive strides. Gait data were pooled from all four paws and used to determine numerous gait dynamic measures including stride length and frequency, variability of stance width and paw area at peak stance.

Locomotion. To accurately measure locomotor behaviors including the total distance traveled and the number of low mobility bouts (defined as remaining continuously in a virtual circle of 15 mm radius for 10 sec), a force-plate actometer (constructed in Dr. Fowler's laboratory, University of Kansas) was used as described previously (Fowler et al., 2001). Mice were placed in a 28 cm×28 cm force-plate actometer for a 30-min recording once a week. When the animal moves on the plate, its movements are sensed by four supporting force transducers positioned at the corners of the plate. The signals are processed by specialized computer algorithms written in house to yield measurements of travel distance and bouts of low mobility.

Rotarod performance. A rotarod apparatus (MED-Associates, St. Albans, Vt.) was used to measure fore- and hindlimb motor coordination and balance. The mice were given a training session at 9 weeks of age (four trials per day for 3 consecutive days) to acclimate them to the rotarod apparatus. During the test period, each mouse was placed on the rotarod with increasing speed, from 4 rpm to 40 rpm in 300 seconds. The latency to fall off the rotarod within this time period was recorded. Mice were tested on the rotorod once a week. Each mouse received two consecutive trials and the mean latency to fall was used in the analysis.

Immunoprecipitation (IP). Mouse brains were bisected mid-saggitally. One half was used in IP, CaM Kinase II Activity and TGase assays. The other half was used for immunohistochemistry assays. To prepare striatal homogenates for IP, a half brain was cut into 300-μm coronal sections with a cryostat (Leica, Nussloch, Germany). Striatum was punched out from five consecutive sections and homogenized in lysate buffer (10 mM Tris-HCl, pH 7.5, 0.14 M NaCl, 1 mM EDTA, and 1:1000 protease inhibitor mixture) in a Tissue Tek homogenizer. Protein concentration was determined using the BCA Protein Assay kit (Pierce Chemical, Rockford, Ill.) and tissue homogenates containing 150 μg of protein were centrifuge at 12,000×g for 5 min at 4° C. to separate the insoluble fraction. After removal of the supernatant, the insoluble fraction was resuspended in 60 μl of 95% formic acid and incubated in a shaking water bath at 37° C. for 40 min. The formic acid was then evaporated using a CentriVap Speed Vacuum (Labconco, Kansas City, Mo.) for 1.5 h at 45° C. The pellets were resuspended in 150 μl of IP wash buffer (10 mM Tris-HCl, pH 7.5, 0.14 M NaCl, and 0.1% Tween 20) and sonicated on ice at 10×5 sec pulse. Immunopurification of proteins containing TGase-catalyzed bonds was performed using 81D4 monoclonal antibody prebound to Sepharose beads (Covalab, Lyon, France) using a protocol developed by Covalab and as described previously (Norlund et al., 1999; Zainelli et al., 2005).

Immunoblot. Immunoaffinity-purified proteins were separated on 10% SDS-polyacrylamide gels and then electrophoretically transferred to nitrocellulose membranes. Membranes were then incubated in blocking buffer (5% nonfat dry milk, 0.1% Tween 20, and 1× TBS) for 1 h at room temperature. Membranes were incubated overnight at 4° C. with primary antibody on a shaker. Primary antibody (Chemicon International, Temecula, Calif.: anti-Huntingtin amino acids 1-82, mouse IgG, 1:500) was diluted in antibody buffer (1% nonfat dry milk, 0.1% Tween 20, and 1× TBS). The next day, membranes were washed with TBS/0.1% Tween 20, and then they were incubated with goat anti-mouse secondary antibody conjugated to horseradish peroxidase (Jackson ImmunoResearch Laboratories Inc., West Grove, Pa.) diluted in antibody buffer. Membranes were washed, and signal was detected using enhanced chemiluminescence Western blotting detection reagents (GE Healthcare, Chalfont, St. Giles, UK). Using Scion Image for Windows (Scion Corporation, Frederick, Md.), immunoblots were quantified by calculating the integrated optical density (IOD) of each protein band on the film.

Evaluation of htt aggregates by immunofluorescence. Sections of frozen mouse brain tissue (20 μm thick) were mounted on glass slides, and then fixed in 4% paraformaldehyde. After fixation, the sections were washed in PBS (pH 7.4), and then nonspecific binding was blocked with 5% normal goat serum (NGS). Sections were incubated overnight with primary antibody: MAB5374 (mouse IgG, 1:100, directed against human huntingtin amino acids 1˜256) in 1% NGS+1× PBS. Next, sections were washed and incubated for 1 h with secondary antibody: goat anti-mouse IgG, Fcγ fragment specific, conjugated to DyLight 649 (1:400) (Jackson ImmunoResearch, West Grove, Pa.). Next, coverslips were mounted on tissue sections with Prolong Gold antifade reagent with DAPI (Invitrogen, Carlsbad, Calif.). As a control for nonspecific labeling with secondary antibody, omission of the primary antibody was also performed on a slide from each case. Htt-aggregate positive nuclei and size of htt-aggregates were measured in each of ten 215×215 μm microscope fields by Olympus IX-81 microscope (Olympus, Japan), in each of five rostrocaudally spaced sections in the striatum of 5 mice from the three groups of R6/2 mice (wild-type littermates did not show htt immunoreactivity). At least 450 nuclei per mouse were obtained and the percentage of nuclei containing htt-aggregates and area of htt-aggregates were calculated by CellProfiler software (Whitehead Institute, Cambridge, Mass.) and a mean value was obtained for each group.

Measurement of striatal volume by Nissl staining. Cryostat sections (20 μm thick) were postfixed in 4% paraformaldehyde, then dehydrated in graded alcohols. After delipidated in 1:1 alcohol/chloroform for 1.5 h, sections were rehydrated through graded alcohols and stained with 0.2% aqueous solution of cresyl violet (Sigma, St. Louis, Mo.) for 5 min, followed by a brief rinse with water and dehydration in graded alcohols. Sections were cleared in xylene (two changes, 5 min each) and cover-slipped with Permount (Fisher Scientific, Fair Lawn, N.J.). The volume of the striatum was measured according to the principle of Cavalieri (Cyr et al., 2005) (volume=s₁d₁+s₂d₂ . . . sₙdₙ, where s=surface area and d=distance between two sections). We considered 15 coronal levels of the striatal sections (from Bregma 1.7 mm, with an interval of 200 μm between the sections) for the volumetric measurement study. Image capturing was performed by using an inverted microscope (Olympus IX-81) coupled to a digital camera (Hamamatsu EMCCD, Japan). The montage image of a coronal brain section was constructed by Slidebook (Intelligent Imaging Innovations, Denver, Colo.). The surface area of striatum in each section was measured using NIH Image J [http://rsbweb.nih.gov/ij/]. Data were expressed as the average Cavalieri volume±SEM (mm³) of 4˜5 mice per group.

TGase II Activity Assay. TGase activity was measured by detecting the incorporation of a biotinylated TGase amine substrate, 5-(biotinamido)pentylamine (Pierce, Rockford, Ill.) into N,N'-dimethylcasein (Calbiochem, San Diego, Calif.) as previously described (Dudek et al., 2009). 96-well Immulon 4 HBX plates (Dynatech, Franklin, Mass.) were coated with 100 μl of N,N′-dimethylcasein (20 mg/ml) in 0.05M sodium bicarbonate, and stored at 4° C. overnight. After the unbound N,N′-dimethylcasein was discarded, the plate was washed with PBS and blocked with nonfat dry milk (2% milk in PBS) for 1 hour at 37° C., followed by 3 washes with PBS. Striatum from the various mice were homogenized in 0.1 M Tris-HCl pH 8.3, 1 mM EDTA, 1 mM PMSF, and protease inhibitor cocktail. Striatal homogenates (15 μg of protein/sample) were added to the plate with 0.1 M Tris-HCl pH 8.5, 0.15 M NaCl, 5 mM DTT, 0.5 mM biotin-labeled pentylamine, and 5 mM CaCl₂. The mixture was incubated for 1 hour at 37° C. The plate was washed with TBS and 0.001% Tween followed by incubation with streptavidin conjugated to HRP (Jackson ImmunoResearch, West Grove, Pa.) for 1 hour at room temperature. After five washes, 1×TMB substrate solution (eBioscience, SanDiego, Calif.) was added and color was allowed to develop for 3 min. Then the reaction was stopped with 3N sulfuric acid and the absorbance was read at 450 nm. For GTP-inhibited TGase enzymatic activity, the assay was performed in the presence of 500 μM GTPγS (Sigma-Aldrich, St Louis, Mo.). Negative controls were run in the absence of pentylamine or dimethylcasein. Each assay also included a standard curve of varying amounts of guinea pig TGase (Sigma, St. Louis, Mo.). Each sample was measured in triplicate. TGase activity was converted to a percentage control based on the mean value for the Vec-WT mice.

### Example Set C Results

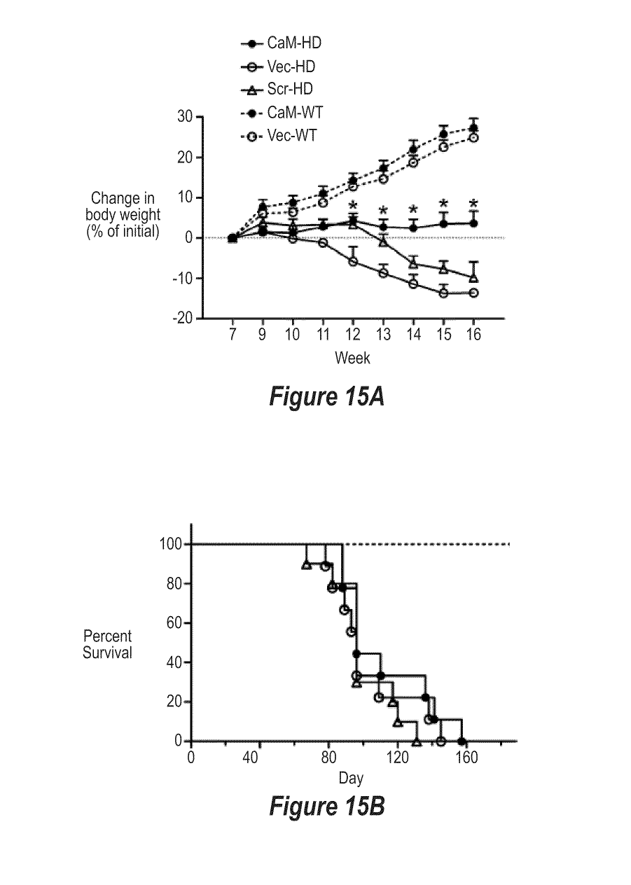

It was found that AAV-mediated delivery of CaM-fragment in R6/2 mice attenuated body weight loss. Body weight was monitored from 7 weeks of age onwards. All groups of mice were of similar initial body weight (22.9±0.3 g, n=10˜14 mice in each group). Changes in body weight were expressed as a percentage of body weight measured at 7 weeks of age (FIG. 15A). Body weight in all groups slowly increased or remained unchanged until week 10. Thereafter, mice in the two wild-type (WT) groups continued gaining body weight. The Vec-HD (R6/2 mice injected with empty vector AAV expressing GFP alone) and Scr-HD mice (R6/2 mice injected with AAV expressing a scrambled version of the CaM-fragment) had profound weight loss over the duration of the observation period, whereas CaM-HD mice (R6/2 mice injected with AAV expressing the CaM-fragment) maintained their body weight, or slightly increased their body weight (FIG. 15A). One-way ANOVA [F_{(4,50)}=82.48; p<0.0001] followed by Bonferroni's multiple comparison test indicated that mice in CaM-HD group had less change in body weight than those in the Vec-HD group at 12-16 weeks of age (p<0.05), and there was no significant difference in body weight change between Scr-HD and Vec-HD at each time point. Similarly, body weight change in the CaM-WT (WT mice injected with AAV expressing the CaM-fragment) was not significantly different from the Vec-WT (WT mice injected with empty vector AAV expressing GFP alone).

It was found that CaM-fragment expression did not significantly increase R6/2 mice survival. Although the first death took place on day 88, 78 and 67 and mean survival time was 112, 102.9, 99.7 days in CaM-HD, Vec-HD and Scr-HD mice, respectively, suggesting a small (about 10%) increase in life span of CaM-HD mice, Kaplan-Meier survival curves demonstrated no statistically significant effect of CaM-fragment expression on the mortality in R6/2 mice (FIG. 1B). By log-rank comparison, the three groups of HD mice did not significantly differ from each other (p>0.05). None of the mice in WT groups died during the observation period (from 6 to 27 weeks of age).

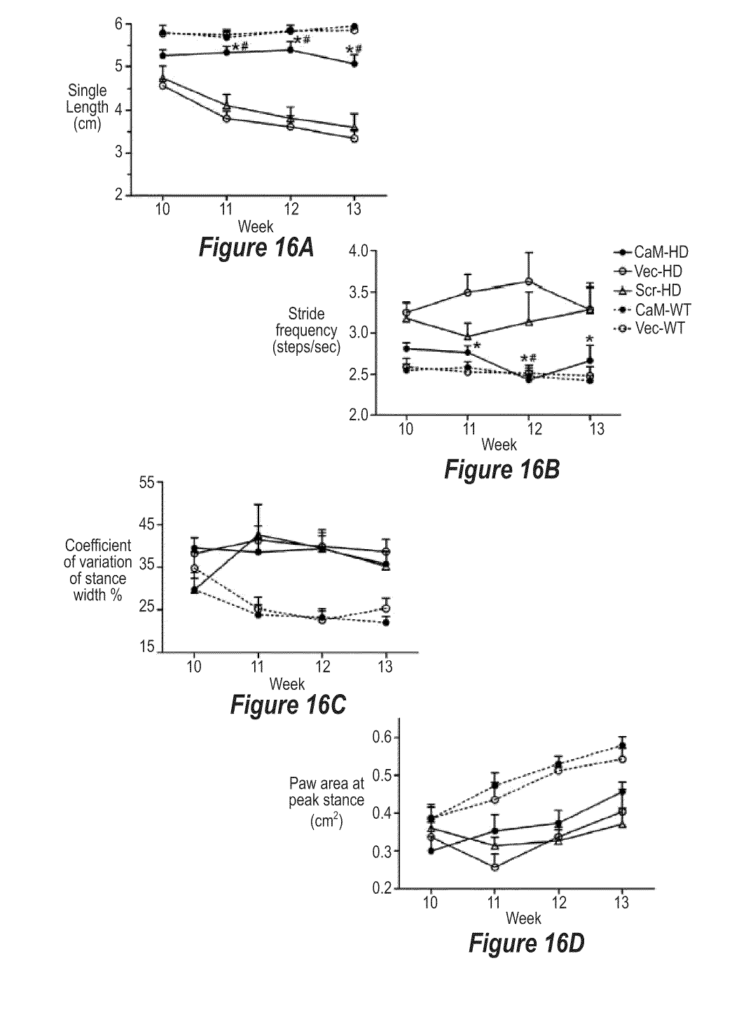

It was found that CaM-fragment expression increases stride length and lowers stride frequency in R6/2 mice. Gait dynamics were characterized using the DigiGait Imaging System. Generally, there is a clear difference between WT mice and Vec-HD or Scr-HD mice starting from week 11 in all the parameters measured, whereas the difference between WT mice and CaM-HD mice is not significant in stride length and frequency. At a speed of 14 cm/s, WT mice maintained a regular alternating gait at a consistent stride length of 5.8±0.3 cm and stride frequency of 2.5±0.1 steps/sec, whereas Vec-HD and Scr-HD mice walked at a gradually reduced stride length and higher stride frequency. For stride length (FIG. 16A), the two-way ANOVA with repeated measures indicated a significant main effect of group [F_{(4,126})=35.77; p<0.0001] and week [F_{(3,126)}=12.12; p<0.0001]. There was also a significant interaction between group and week [F_{(12,126)}=5.71; p<0.0001]. The post hoc Bonferroni test indicated that stride length of CaM-HD mice is indistinguishable from the two HD control groups at 10 weeks of age, but CaM-HD mice displayed a longer stride length as compared to Vec-HD or Scr-HD mice at week 11˜13. Stride length of CaM-HD mice was not significantly shorter than WT mice until at week 13. For stride frequency (FIG. 16B), two-way ANOVA indicated significant differences among groups [F_{(4,126)}=11.99; p<0.0001], but there is no significant effect of week [F_{(3,126)}=0.202; p=0.895] or interaction between group and week [F_{(12,126)}=1.368; P=0.190]. The Bonferroni test indicated that CaM-HD mice had a significantly lower stride frequency than Vec-HD at week 11-13. Although Vec-HD and Scr-HD were statistically indistinguishable at any time point, there was a statistically significant difference between CaM-HD and Scr-HD at week 12. There was no significant difference between CaM-HD and WT mice in stride frequency. Analysis of some other gait dynamics revealed differences between genotypes. For example, the measure of stance width variation between steps was greater (FIG. 16C) and paw area at peak stance was smaller (FIG. 16D) in HD mice than in WT mice, but the CaM-fragment had no beneficial effects on these gait indices. For stance width variation, two-way ANOVA followed by Bonferroni test indicated significant differences between HD and WT groups [F_{(4,126)}=13.61; p<0.0001], but no significant differences among HD groups or WT groups (P>0.05). There is no significant effect of week [F_{(3,126)}=1.21; p=0.31] although interaction between group and week is significant [F_{(12,126)}=1.97; P=0.03]. Similar results were obtained in the analysis of paw area at peak stance [Group factor: F_{(4,126)}=10.42; p<0.0001], whereas week effect is significant [F_{(3,126)}=13.68; and interaction between group and week is not [F_{(12,126)}=1.55; P=0.11].

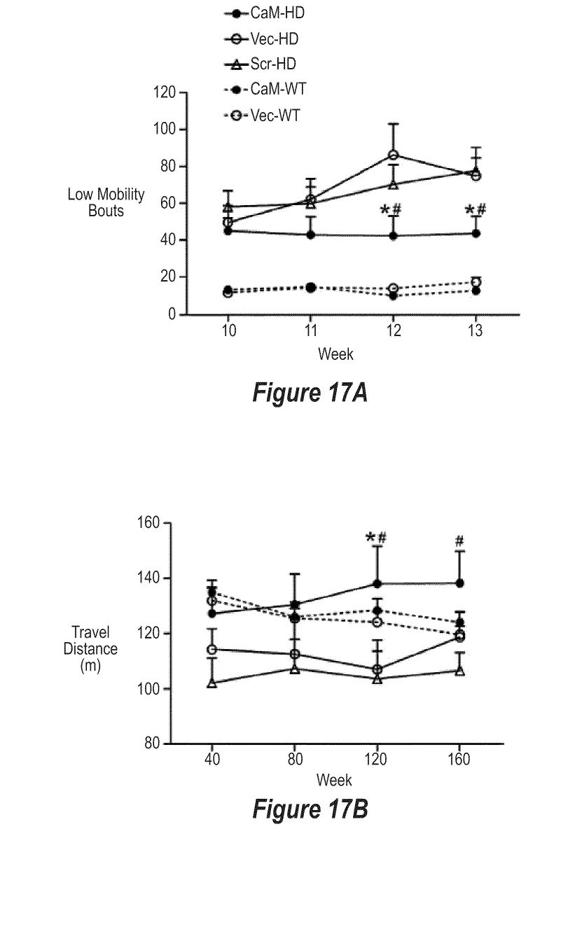

It was found that CaM-HD mice had fewer low mobility bouts and longer travel distance than Scr-HD or Vec-HD mice. Force-plate actometers were used to measure the locomotor activity of R6/2 and WT mice. The number of low mobility bouts (LMB, defined as remaining continuously in a virtual circle of 15 mm radius for 10 sec) is presented in FIG. 17A. Two-way ANOVA with repeated measures indicated a significant effect of group [F_{(4,144)}=27.75, p<0.001] and a significant effect of week [F_{(3, 144)}=3.179, p<0.05], but the interaction between group and week was not significant. The number of LMB gradually increased in HD mice without CaM-fragment expression, whereas CaM-HD mice, similar to WT mice, maintained LMB at a relatively lower level. At week 12 and 13, the Bonferroni post hoc test detected significantly fewer LMB in CaM-HD mice as compared to either Scr-HD or Vec-HD mice. Total travel distance in a 30 min session provided another index of locomotor activity of HD mice (FIG. 17B). Two-way ANOVA with repeated measures indicated a significant effect of group [F_{(4,138)}=3.40, p=0.02], but the effect of week [F_{(3,138)}=0.17, p=0.92] and interaction between group and week [F_{(12,138)}=1.12, p=0.35] were no significant. Consistent with reduced LMB in HD mice treated with CaM-fragment, CaM-fragment expression also increased the locomotor activity in HD mice as indicated by a significantly greater travel distance in CaM-HD mice than in either Scr-HD (at week 12, 13) or Vec-HD mice (at week 12). There was no significant difference between CaM-HD and WT mice in distance traveled in 30 min.

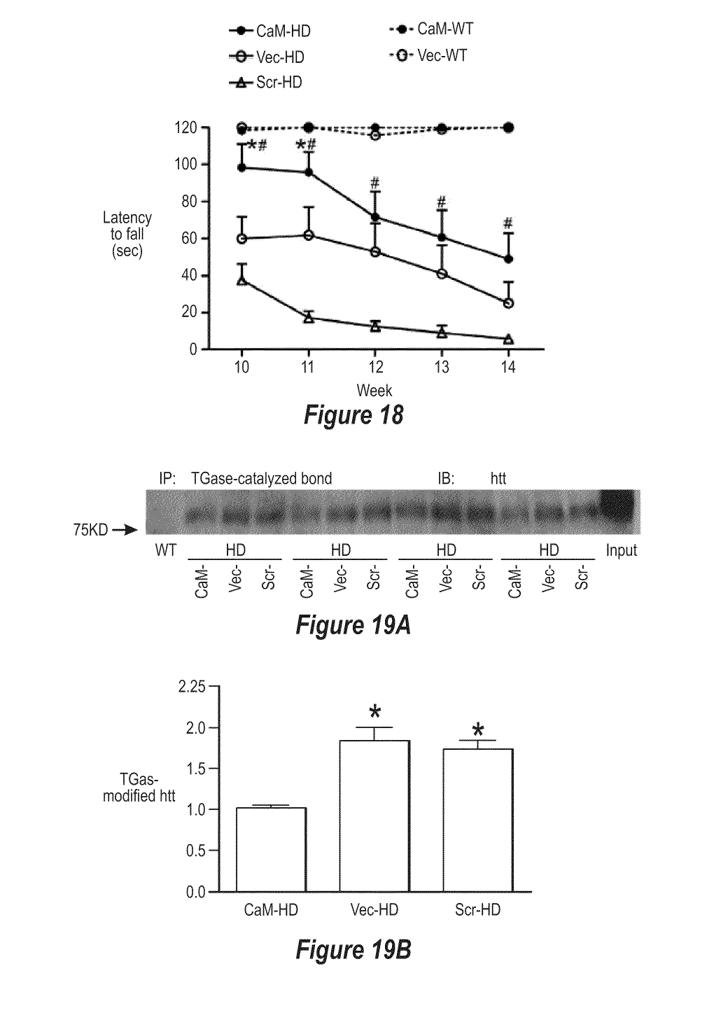

It was found that CaM-fragment improved performance on the rotarod in R6/2 mice. Rotarod was used to measure fore- and hindlimb motor coordination and balance starting at 10 weeks of age (i.e., three weeks after the AAV injections). At ten weeks of age, the two groups of WT mice were able to stay on the rod during the 300 sec time period measured, whereas none of the three groups of HD mice could finish the test without falling in 300 sec. Even at this early time point after injection of the AAV, the mean latency to fall was significantly differed among the HD groups, 245.9±32.0, 150.2±29.5, 98.1±19.8 sec in CaM-HD, Vec-HD and Scr-HD mice, respectively. After 10 weeks of age, there was a progressive decline in performance of all HD mice, but the CaM-HD mice still achieved better performance than the other two HD groups (FIG. 18). Using analysis using two-way ANOVA with repeated measurements, we found a significant main effect of group [F_{(4,188)}=49.35, p<0.0001], a significant main effect of week [F_{(4,188)}=18.86, p<0.0001] and a significant interaction between group and week [F_{(16,188)}=4.714, p<0.0001] on latency to fall off the rotarod. Bonferroni post hoc test indicated that CaM-HD mice had longer latency to fall as compared to Scr-HD mice at 10-14 weeks of age and Vec-HD mice at 10-11 weeks of age (p<0.05).

It was found that CaM-fragment expression reduced TGase-modified htt in R6/2 mice striatum. Cell culture studies indicated that AAV-mediated CaM-fragment expression can significantly inhibit the formation of TGase-modified htt (Dudek et al., 2009). Here we used striatal homogenates from HD or WT mice to detect TGase-catalyzed cross-linking of htt. Immunopurification of proteins containing ε-(γ-glutamyl)lysine bonds was performed using 81D4 mAb prebound to Sepharose beads. TGase-modified htt was then detected on immunoblots as shown in the FIG. 19A. TGase-modified htt was only detected in HD but not WT mice. One-way ANOVA [F_{(2,15)}=18.44, p<0.0001] followed by Bonferroni's comparison test found there was a significant reduction (by approximate 50%) in TGase-modified htt in the striatum of CaM-HD mice as compared to Scr-HD or Vec-HD mice (FIG. 19B).

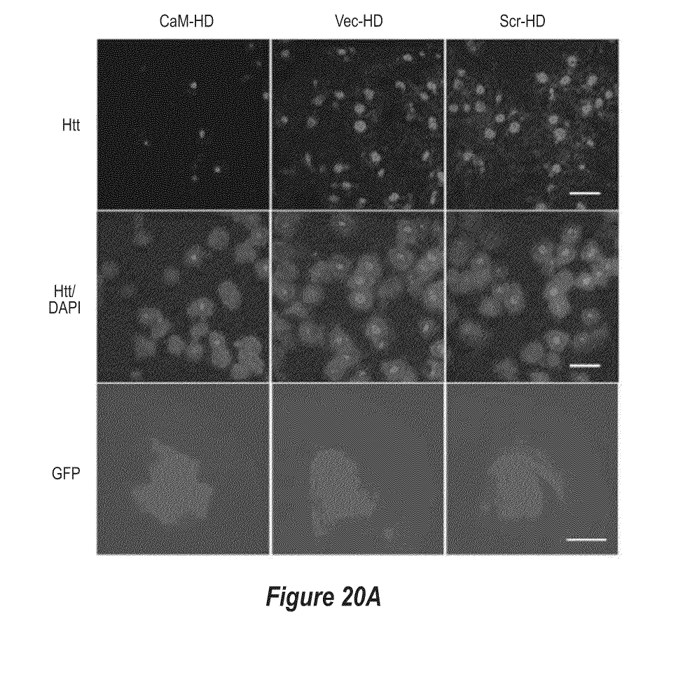

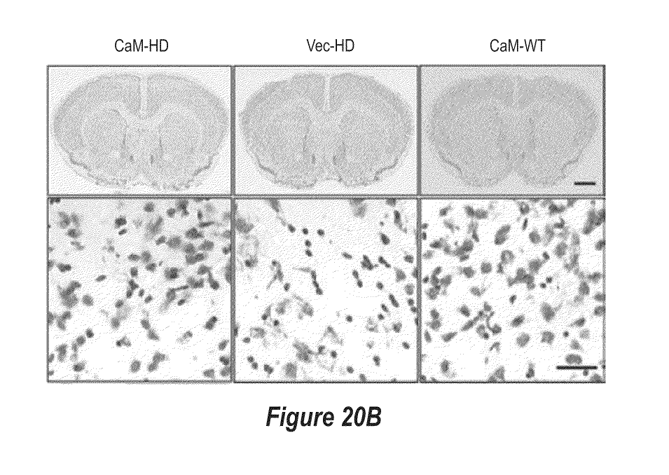

It was found that CaM-fragment expression decreases the percentage of htt-positive nuclei and the size of htt aggregates in the striatum of R6/2 mice. Striatal intranuclear and neuropil inclusions containing mutant htt are prominent neuropathological hallmarks of HD and may play an important role in disease progression. In R6/2 mice, intranuclear aggregates are much larger than neuropil aggregates and amenable to quantification, so we examined the effect of CaM-fragment on intranuclear htt aggregates in the striatum of R6/2 mice. In CaM-HD mice, the percentage of htt-positive nuclei was lower and the size of intranuclear aggregates appeared smaller than in the control virus-treated HD mice (FIG. 20A). These observations were confirmed by quantification of nuclear aggregates (Table 1).

TABLE 1
Quantitative analysis of neuropathology

| | Cam-HD | Vec-HD | Scr-HD | CaM-WT | Vec-WT |
|---|---|---|---|---|---|
| Percentage of nuclei | 71.7 ± 5.6 | 85.1 ± 2.1* | 89.5 ± 0.8^{a*} | | |
| containing htt | | | | | |
| aggregates (%) | | | | | |
| Size of intranuclear htt | 248.4 ± 27.1 | 378.1 ± 31.5* | 391.0 ± 28.2^{b*} | | |
| aggregates (μm²) | | | | | |
| Striatal volume (mm³) | 14.5 ± 0.3 | 13.8 ± 0.7 | 13.6 ± 0.3 | 17.0 ± 0.8^{#} | 17.2 ± 0.4^{c#} |

^{a}F_{(2,12)} = 6.83,
^{b}F_{(2,12)} = 7.38,
^{c}F_{(4,17)} = 11.77.
*p < 0.05 as compared to CaM-HD (one-way ANOVA, n = 5 in each group at 13-14 weeks of age).
^{#}p < 0.05 as compared to CaM-HD (one-way ANOVA, n = 4-5 in each group at 14-20 weeks of age for HD mice, at 27-28 weeks of age for WT mice).

We found a significant reduction in the percentage of striatal nuclei containing htt-aggregates (approximately 18%) and the nuclear aggregate size (approximately 35%) in CaM-HD mice as compared with either Vec- or Scr-HD mice. GFP fluorescence was observed in coronal brain sections from AAV-injected mice, indicating a widespread infection and expression of CaM-fragment in the striatum (FIG. 20A). Both human HD and R6/2 mouse brains are characterized by atrophy of the striatum. To exam whether the CaM-fragment delivery decreases gross striatal atrophy, we used Nissl staining and the Cavalieri principle to estimate striatal volumes. There was no statistically significant difference in mean striatal volumes between CaM-HD and the two groups of control HD mice. However, all three groups of HD mice had significantly smaller striatal volumes as compared with WT littermates (FIG. 20B, Table 1). As expected, striatal neuronal atrophy was observed in HD mice in high-power images.

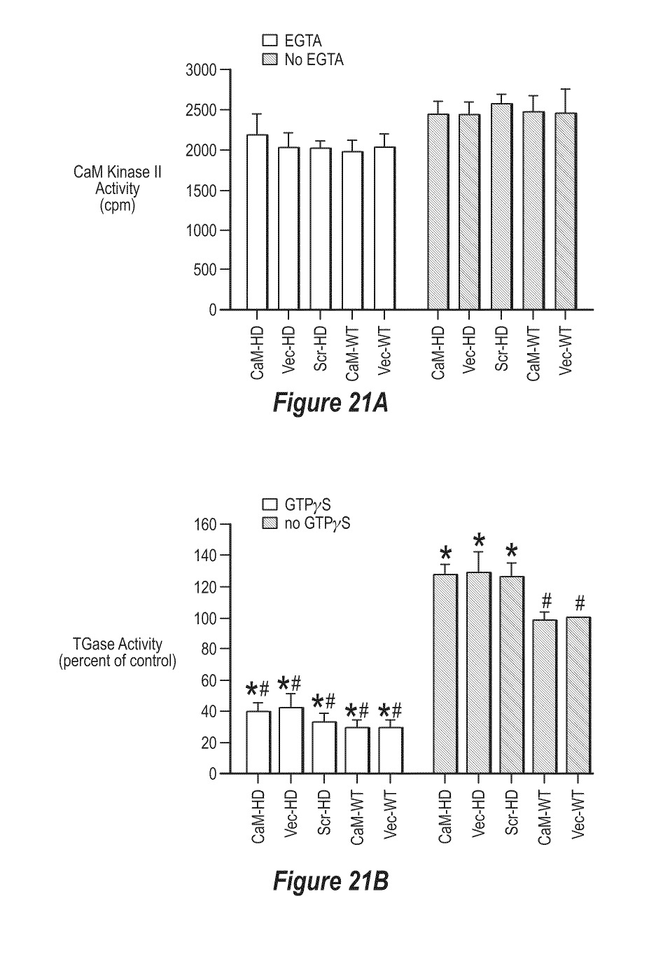

It was found that CaM Kinase II activity was not affected by CaM-fragment expression. To test if expression of the CaM-fragment would interfere with other CaM-dependent enzymes, we measured the activity of calmodulin-dependent protein kinase II (CaM kinase II) in mouse striatal homogenates. We found no significant differences in CaM kinase II activity among the various HD or WT mouse groups in either the presence or absence of EGTA, a calcium chelator (FIG. 21A). However, EGTA caused a general decrease in CaM kinase II activity in all mouse groups. Two-way ANOVA indicated a significant main effect of EGTA [F_{(1,50)}=72.12; p<0.0001, but there was no significant effect of mouse group [F_{(4,50)}=0.543; p=0.705] or an interaction between EGTA and group [F_{(4,50)}=0.966; p=0.435].

It was found that CaM-fragment did not change total TGase activity. In order to explore whether the reduced TGase-modified htt in CaM-HD mice is due to a substrate-dependent inhibition or a general decrease in TGase enzymatic activity, we compared total TGase activity in the striatum among the different groups of mice. There is a significant increase in TGase activity in the three groups of HD mice as compared to CaM-WT or Vec-WT mice (p<0.01). Expression of the CaM-fragment did not have an effect on TGase activity in either HD or WT mice. In the presence of GTPγS, which specifically inhibits TGase activity, TGase activity is dramatically reduced (at least 60%) in all groups (FIG. 21B). Two-way ANOVA indicated a significant main effect of GTPγS [F_{(1,50)}=428.149; p<0.0001] and mouse group [F_{(4,50)}=5.535; p<0.01], and there was a significant interaction between GTPγS and mouse group [F_{(4,50)}=5.032; p<0.01]. The activity of guinea pig liver TGase was measured in each assay in the range from 0.01 to 0.5 milliunits/ml and resulted in a linear correlation with an R-square value from 0.95 to 0.99. The activity of TGase in the striatal samples fell within this linear range for guinea pig liver TGase.

Striatal delivery of CaM-fragment (e.g., CaM-peptide) significantly delayed the onset of movement abnormalities and improved motor function as measured by analysis of gait, rotarod performance and locomotor behavior. Striatal injection of AAV-CaM-fragment conferred significant improvements in stride length and frequency measurements, and a mild, insignificant increase in paw area at peak stance in R6/2 mice. Three weeks after CaM-fragment expression, R6/2 mice started to show significantly improved rotarod performance and this beneficial effect was maintained from 10 weeks of age onward.

The present studies provide, for the first time, in vivo evidence that CaM-peptide has significant efficacy in improving the behavioral deficits and neuropathological phenotype in the HD animal model. The positive effects of CaM-peptide in R6/2 mice provide further evidence that CaM-regulated TGase modification of htt may contribute to HD pathogenesis. More importantly, these studies have identified a novel therapeutic strategy for treating HD patients.

1. An artificial polypeptide for inhibiting a mutant huntingtin protein from interacting with a calmodulin protein, the polypeptide comprising:
a polypeptide sequence having an amino acid sequence of a portion of calmodulin or analog or derivative thereof, said polypeptide sequence being capable of interacting with mutant huntingtin so as to inhibit interactions between mutant huntingtin and calmodulin.

2. A polypeptide as in claim 1, wherein the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof.

3. A polypeptide as in claim 2, wherein the polypeptide having SEQ ID NO: 1 is truncated by up to 15 amino acids from the N-terminus or the C-terminus. NO: 1.

4. A polypeptide as in claim 2, wherein the polypeptide consists of SEQ ID NO: 1.

5. A pharmaceutical composition comprising:
the artificial polypeptide sequence of claim 1; and
a pharmaceutically acceptable carrier.

6. A nucleic acid encoding for the artificial polypeptide of claim 1.

7. A cell comprising a mutant huntingtin protein bound to the artificial polypeptide of claim 1.

8. A viral particle comprising a nucleic acid encoding for the artificial polypeptide of claim 1.

9. A viral particle as in claim 8, wherein the viral particle is from an adeno-associated virus.

10. A viral particle as in claim 8, wherein the artificial polypeptide includes SEQ ID NO: 1.

11. A method of treating, inhibiting, and/or preventing Huntington's Disease, the method comprising:
providing an substance being capable of interacting with mutant huntingtin so as to inhibit interactions between mutant huntingtin and calmodulin; and
administering the substance to a subject having or susceptible to Huntington's Disease such that the substance interacts with the mutant huntingtin so as to inhibit the mutant huntingtin protein from interacting with a calmodulin protein.

12. A method as in claim 11, wherein said the substance is an artificial polypeptide sequence having an amino acid sequence of a portion of calmodulin or analog or derivative thereof.

13. A method as in claim 12, wherein the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof.

14. A method as in claim 13, wherein the polypeptide having SEQ ID NO: 1 is truncated by up to 15 amino acids from the N-terminus or the C-terminus.

15. A method as in claim 11, wherein the polypeptide consists of SEQ ID NO: 1.

16. A method of inhibiting calmodulin from interacting with a mutant huntingtin protein, the method comprising:
providing a mutant huntingtin protein having a polyglutamine sequence in the presence of a calmodulin protein; and
contacting the mutant huntingtin protein with a substance capable of interacting with the mutant huntingtin so as to inhibit interactions between the mutant huntingtin and calmodulin proteins.

17. A method as in claim 16, wherein said the substance is an artificial polypeptide sequence having an amino acid sequence of a portion of calmodulin or analog or derivative thereof.

18. A method as in claim 17, wherein the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof.

19. A method as in claim 17, wherein the polypeptide having SEQ ID NO: 1 is truncated by up to 15 amino acids from the N-terminus or the C-terminus.

20. A method as in claim 17, wherein the polypeptide consists of SEQ ID NO: 1.

21. A method as in claim 15, wherein the substance bound to the mutant huntingtin protein inhibits the mutant huntingtin protein from interacting with a transglutaminase.

22. A method as in claim 15, wherein the mutant huntingtin protein is located within a cell.

23. A method as in claim 15, wherein the mutant huntingtin protein is located within a subject having Huntington's disease.

24. A method for screening for a substance that inhibits mutant huntingtin from interacting with calmodulin, the method comprising:
providing a mutant huntingtin protein or a mutated portion thereof having a polyglutamine;
contacting the mutant huntingtin protein or mutated portion thereof with a substance; and
determining whether or not the substance inhibits the mutant huntingtin protein or mutated portion thereof from interacting with the calmodulin protein.

25. A method as in claim 24, wherein the contacting is conducted within a cell.

26. A method as in claim 24, wherein the determining includes contacting the mutant huntingtin protein or mutated portion thereof with calmodulin-agarose.

27. A method as in claim 24, further comprising providing a cell expressing the mutant huntingtin or mutated portion thereof.

28. A method as in claim 27, said cell also expressing a transglutaminase.

29. A method as in claim 28, further comprising:
lysing the cell; and
mixing the cell lysate with calmodulin-agarose.

30. A method for inhibiting transglutaminase from modifying a mutant huntingtin protein, the method comprising;
providing a mutant huntingtin protein having a polyglutamine sequence; and
contacting the mutant huntingtin protein with a substance capable of interacting with the mutant huntingtin so as to inhibit a transglutaminase from modifying the mutant huntingtin.

31. A method as in claim 30, wherein said the substance is an artificial polypeptide sequence having an amino acid sequence of a portion of calmodulin or analog or derivative thereof.

32. A method as in claim 31, wherein the polypeptide sequence includes a sequence of KDTDSEEEIREAFRVFDKDGNGYISAAELRHVMTNLGEKLTDEEV (SEQ ID NO: 1) or a portion thereof analog thereof or derivative thereof.

33. A method as in claim 32, wherein the polypeptide having SEQ ID NO: 1 is truncated by up to 15 amino acids from the N-terminus or the C-terminus.

34. A method as in claim 32, wherein the polypeptide consists of SEQ ID NO: 1.


SEQUENCE LISTING (32 sequences)

SEQ ID NO: 1 - protein, 45 aa, Artificial Sequence
  Description of Artificial Sequence Synthetic polypeptide
KDTDSEEEIREAFRVFDKDGNGYISAAELRHVMTNLGEKLTDEEV

SEQ ID NO: 2 - protein, 40 aa, Artificial Sequence
  Description of Artificial Sequence Synthetic polypeptide
EEEIREAFRVFDKDGNGYISAAELRHVMTNLGEKLTDEEV

SEQ ID NO: 3 - protein, 40 aa, Artificial Sequence
  Description of Artificial Sequence Synthetic polypeptide
KDTDSEEEIREAFRVFDKDGNGYISAAELRHVMTNLGEKL

SEQ ID NO: 4 - protein, 35 aa, Artificial Sequence
  Description of Artificial Sequence Synthetic polypeptide
EEEIREAFRVFDKDGNGYISAAELRHVMTNLGEKL

SEQ ID NO: 5 - protein, 35 aa, Artificial Sequence
  Description of Artificial Sequence Synthetic polypeptide
EAFRVFDKDGNGYISAAELRHVMTNLGEKLTDEEV

SEQ ID NO: 6 - protein, 35 aa, Artificial Sequence
  Description of Artificial Sequence Synthetic polypeptide
KDTDSEEEIREAFRVFDKDGNGYISAAELRHVMTN

SEQ ID NO: 7 - protein, 30 aa, Artificial Sequence
  Description of Artificial Sequence Synthetic polypeptide
EEEIREAFRVFDKDGNGYISAAELRHVMTN

SEQ ID NO: 8 - protein, 30 aa, Artificial Sequence
  Description of Artificial Sequence Synthetic polypeptide
EAFRVFDKDGNGYISAAELRHVMTNLGEKL

SEQ ID NO: 9 - protein, 30 aa, Artificial Sequence
  Description of Artificial Sequence Synthetic polypeptide
FDKDGNGYISAAELRHVMTNLGEKLTDEEV

SEQ ID NO: 10 - protein, 30 aa, Artificial Sequence
  Description of Artificial Sequence Synthetic polypeptide
KDTDSEEEIREAFRVFDKDGNGYISAAELR

SEQ ID NO: 11 - protein, 25 aa, Artificial Sequence
  Description of Artificial Sequence Synthetic peptide
EEEIREAFRVFDKDGNGYISAAELR

SEQ ID NO: 12 - protein, 25 aa, Artificial Sequence
  Description of Artificial Sequence Synthetic peptide
FDKDGNGYISAAELRHVMTNLGEKL

SEQ ID NO: 13 - protein, 20 aa, Artificial Sequence
  Description of Artificial Sequence Synthetic peptide
EAFRVFDKDGNGYISAAELR

SEQ ID NO: 14 - protein, 20 aa, Artificial Sequence
  Description of Artificial Sequence Synthetic peptide
FDKDGNGYISAAELRHVMTN

SEQ ID NO: 15 - protein, 15 aa, Artificial Sequence
  Description of Artificial Sequence Synthetic peptide
FDKDGNGYISAAELR

SEQ ID NO: 16 - DNA, 36 bp, Artificial Sequence
  Description of Artificial Sequence Synthetic primer
agggctaggtaccgaatcatggctgatcagcgtacc

SEQ ID NO: 17 - DNA, 40 bp, Artificial Sequence
  Description of Artificial Sequence Synthetic primer
ccctctagactcgagcggccgccacatttttctagccatc

SEQ ID NO: 18 - DNA, 48 bp, Artificial Sequence
  Description of Artificial Sequence Synthetic primer
agggctaggtaccgaatcatgaaggacacagacagtgaggaggagatc

SEQ ID NO: 19 - DNA, 42 bp, Artificial Sequence
  Description of Artificial Sequence Synthetic primer
ccctctagactcgagcggccgccattttgcagtcatcatctg

SEQ ID NO: 20 - DNA, 42 bp, Artificial Sequence
  Description of Artificial Sequence Synthetic primer
agggctaggtaccgaatcatggaagcagagctgcaggatatg

SEQ ID NO: 21 - DNA, 39 bp, Artificial Sequence
  Description of Artificial Sequence Synthetic primer
ccctctagactcgagcggccgccacacctcatcggtcag

SEQ ID NO: 22 - protein, 10 aa, Artificial Sequence
  Description of Artificial Sequence Synthetic peptide
EQKLISEEDL

SEQ ID NO: 23 - protein, 45 aa, Artificial Sequence
  Description of Artificial Sequence Synthetic polypeptide
GDTVEREKDAYNSLEGFDNTIHTLRADIGMVEEVAKSKRDEEFLE

SEQ ID NO: 24 - DNA, 36 bp, Artificial Sequence
  Description of Artificial Sequence Synthetic primer
actcgactcgagatcatgaaggacacagacagtgag

SEQ ID NO: 25 - DNA, 37 bp, Artificial Sequence
  Description of Artificial Sequence Synthetic primer
actcgagaattcctacacctcctcatcggtcagcttc

SEQ ID NO: 26 - DNA, 36 bp, Artificial Sequence
  Description of Artificial Sequence Synthetic primer
actcgaggtaccttacttgtacagctcgtccatgcc

SEQ ID NO: 27 - DNA, 35 bp, Artificial Sequence
  Description of Artificial Sequence Synthetic primer
actcgactcgagatcatgggcgataccgtggaacg

SEQ ID NO: 28 - DNA, 36 bp, Artificial Sequence
  Description of Artificial Sequence Synthetic primer
actcgagaattcctattccagaaattcttcatcacg

SEQ ID NO: 29 - protein, 148 aa, Unknown
  Description of Unknown Wild-type calmodulin polypeptide
ADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 30 - protein, 76 aa, Artificial Sequence
  Description of Artificial Sequence Synthetic polypeptide
ADQLTEEQIAEFKEAFSLFDKDGDGTITTKELGTVMRSLGQNPTEAELQDMINEVDADGNGTIDFPEFLTMMARKM

SEQ ID NO: 31 - protein, 78 aa, Artificial Sequence
  Description of Artificial Sequence Synthetic polypeptide
TEAELQDMINEVDADGNGTIDFPEFLTMMARKMKDTDSEEEIREAFRVFDKDGNGYISAAELRHVMTNLGEKLTDEEV

SEQ ID NO: 32 - protein, 73 aa, Artificial Sequence
  Description of Artificial Sequence Synthetic polypeptide
MKDTDSEEEIREAFRVFDKDGNGYISAAELRHVMTNLGEKLTDEEVDEMIREADIDGDGQVNYEEFVQMMTAK